(19) United States
(12) Patent Application Publication
(10) Pub. No.: US 2026/0125650 A1
(43) Pub. Date: May 7, 2026

(54) METHODS AND COMPOSITIONS FOR GENERATING HEPATOCYTES

(71) Applicant: Trailhead Biosystems Inc., Beachwood, OH (US)

(72) Inventors: Michael A. BUKYS, Lakewood, OH (US); Xiaoguang FANG, Solon, OH (US); Faruk SENTURK, Cleveland Heights, OH (US)

(21) Appl. No.: 19/348,925

(22) Filed: Oct. 3, 2025

Related U.S. Application Data
(60) Provisional application No. 63/704,430, filed on Oct. 7, 2024.

(51) Int. Cl. C12N 5/071

(52) U.S. Cl. CPC C12N 5/067; C12N 2500/24; C12N 2500/44; C12N 2501/115; C12N 2501/12; C12N 2506/45

(57) ABSTRACT

Methods for generating hepatocytes from induced pluripotent stem cells (iPSCs) are provided using chemically-defined culture media in a four-stage culture protocol. Culture media and isolated cell populations are also provided.

### BACKGROUND OF THE INVENTION

Hepatocytes are cells of the main parenchymal tissue of the liver that make up the majority of the liver's mass. Hepatocytes have several physiological roles and can be used for research, drug development, and tissue engineering of liver tissue.

Hepatocytes can be obtained from induced pluripotent stem cells (IPSCs). Early approaches for obtaining hepatocytes from iPSCs involve screening of individual components that drive the differentiation, which results in a process optimization that does not consider the synergistic effects among the components. There is a need for efficient and robust methods and compositions for controlled differentiation of stem cells towards a hepatocyte fate, which takes into account the combinatorial effects of the added components.

### SUMMARY OF THE INVENTION

This disclosure provides methods of generating hepatocytes from induced pluripotent stem cells (iPSCs), culture media, and other compositions for use in such methods. Hepatocytes can be obtained from iPSCs in a five-step, four-stage, 24-day protocol that sequentially generates endodermal cells, midgut cells, hepatoblasts, and hepatocytes. The methods use chemically defined culture media that allow for generation of endodermal cells within two days of culture, midgut cells within seven days of culture, hepatoblasts within 17 days of culture, and hepatocytes within 24 days of culture.

The defined culture media used to obtain the different types of cells comprise small molecule agents that either agonize or antagonize particular signaling pathway activity in the pluripotent stem cells such that differentiation along the hepatocyte lineage is promoted, leading to cellular maturation and expression of hepatocyte-associated biomarkers, including but not limited to DLK1, GPC3, CYP3A7 and FXR, CAR, PXR, ASS, ARG1, FGL1, ORM1, CYP2C9, and CYP3A4. The methods of the disclosure use culture media for differentiation that comprise different components than those of earlier protocols. The methods of the disclosure also have the advantage that the use of small molecule agents in the culture media allows for precise control of the culture components and the time needed for differentiation to hepatocytes is significantly shortened compared to prior art protocols, due to the synergistic effects of the small molecule agents in the culture media.

Accordingly, in one aspect, the disclosure pertains to a method of generating a population of hepatocytes. The method includes the steps of:
(a) culturing iPSCs in a culture medium comprising a glycogen synthase kinase 3 beta (GSK3β) inhibitor for 12-120 hours to obtain a first population of endodermal cells;
(b) culturing the first population of endodermal cells from step (a) in a culture medium lacking a GSK3ß inhibitor for 12-120 hours to obtain a second population of endodermal cells;
(c) culturing the second population of endodermal cells from step (b) in a culture medium comprising a histone deacetylase (HDAC) inhibitor, a retinoic acid (RA) agonist, and a fibroblast growth factor receptor (FGFR; e.g., FGFR1) agonist for 24-240 hours to obtain a population of midgut cells;
(d) culturing the population of midgut cells in a culture medium comprising a glucocorticoid, a cAMP response element-binding protein (CREB) agonist, an antioxidant, a Wnt antagonist, a transforming growth factor beta (TGF-β) antagonist, and a hepatocyte growth factor (HGF) agonist for at least 48 hours to obtain a population of hepatoblasts; and
(e) culturing the population of hepatoblasts in a culture medium comprising an HDAC inhibitor, a glucocorticoid, a CREB agonist, an antioxidant, a Wnt antagonist, a TGF-β antagonist, an HGF agonist, a Notch antagonist, a bone morphogenetic protein (BMP) antagonist, and a hepatocyte function enhancer for at least 48 hours to obtain a population of hepatocytes.

In some embodiments, the GSK36 inhibitor is selected from the group consisting of CHIR99021, CHIR98014, CHIR98023, 3F8, A 1070722, AR-A014418, BIO, BIO-acetoxime, 6-BIO, Indirubin-3′-oxime, Alsterpaullone, 1-Azakenpaullone, Cazpaullone, Kenpaullone, Aloisine A, SB 216763, SB 415286,SB41528, SAR502250, TC-G 24, TWS119, LY2090314, AT7519, KY19382, AZD1080, AZD2858, Hymenialdisine, Debromohymenialdisine, Dibromocantherelline, Meridianine A, NSC 693868, IM-12,IMID1, IMID2, VP2.51, VP2.54, BIP-135, JGK-263, MMBO, TCS2002, PF-367, BRD0705, BRD3731, AF3581, TDZD-8, NP00111, Tideglusib (NP031112), NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, and 4-4. In some embodiments, the GSK36 inhibitor is CHIR99021. In some embodiments, CHIR99021 is present in the culture medium at a concentration of 100 nM-10 μM.

In some embodiments, the HDAC inhibitor in each of steps (c) and (e) is independently selected from the group consisting of Vorinostat, Sodium Butyrate, Belinostat, Dacinostat, Droxinostat, Domatinostat, Entinostat, Ricolinostat, Resminostat, Abexinostat, Givinostat, Ivaltinostat, Panobinostat, Pracinostat, Quisinostat, Mocetinostat, Nanatinostat, Nexturastat A, Tefinostat, Tucidinostat, Fimepinostat, Citarinostat, Zabadinostat, GSK3117391, CUDC-101, AR-42, M344, Scriptaid, Sulforaphane, SR-4370, MC1568, CAY10603, CAY10683, RG2833, RGFP966, Cpd60, BRD3308,Tasquinimod, BML-210, LMK-235, BRD73954, PCI-34051, TMP195, TMP269, NKL22, TH34, SIS17, WT-161,ACY-738, BG45, CBHA, Pyroxamide, NCH-51, NCH-31, KD 5170, TCS HDAC6 20b, NSC 3852,NSC 69603, NSC 86371, NSC 305819, MS-27-275, Trapoxin A, Trapoxin B, Romidepsin, Apicidin, Trichostatin A, ACY-775, Tubastatin A, Tubacin, SKLB-23bb, HPOB, Curcumin, UF010, tc-H 106, Splitomicin, Raddeanin A, Depudecin, Tacedinaline, Isoguanosine, Parthenolide, Tinostamustine, Sodium Phenylbutyrate, Valproic Acid, Butyric acid, Phenylbutyric acid, 4-Phenylbutyric Acid, Divalproex Sodium, Sinapinic Acid, Suberohydroxamic Acid, Biphenyl-4-sulfonyl chloride, Thiophene Benzamide, Nicotinamide, Dihydrocoumarin, Naphthopyranone, and 2-Hydroxynaphthaldehyde. In some embodiments, the HDAC inhibitor in step (c) is Vorinostat. In some embodiments, Vorinostat is present in the culture medium at a concentration of 10 nM- 5 μM. In some embodiments, the HDAC inhibitor in step (e) is Sodium Butyrate. In some embodiments, Sodium Butyrate is present in the culture medium at a concentration of 10 nM- 5 μM.

In some embodiments, the RA agonist is selected from the group consisting of TTNPB, ATRA, 9-cis-Retinoic Acid, Adapalene, Tretinoin, WYC-209, DC271, Acitretin, Arotinoid, AGN190168,AGN205327, LGD1550, Ch55, AM580 (CD336), CD2081, BMS 753, Tamibarotene, AGN194078,AGN195183, AGN193836, CD2314, CD2019, CD666, C286, BMS 641, AC-55649, AC261066, KCL-286,CD1530, CD437, CD2325, BMS 189961, BMS 270394, BMS 961, Trifarotene, and Palovarotene. In some embodiments, the RA agonist is TTNPB. In some embodiments, TTNPB is present in the culture medium at a concentration of 5 nM-2 μM.

In some embodiments, the FGFR (e.g., FGFR1) agonist is selected from the group consisting of FGF2, SUN11602, FGF1, FGF3, FGF4, FGF5, FGF6, FGF8, FGF10, FGF17, FGF19, FGF20, FGF21, FGF22, and FGF23. In some embodiments, the FGFR (e.g., FGFR1) agonist is FGF2. In some embodiments, FGF2 is present in the culture medium at a concentration of 500 pg/ml-500 ng/ml.

In some embodiments, the glucocorticoid in steps (d) and (e) is independently selected from the group consisting of Hydrocortisone 21-hemisuccinate, Dexamethasone, Dexamethasone Acetate, Hydrocortisone, Cortisone, Prednisone, Prednisone Acetate, Meprednisone, Prednisolone, Methylprednisolone, Methylprednisolone Acetate, Fluprednisolone, Betamethasone, Paramethasone, Triamcinolone, Deflazacort, Fludrocortisone, Fludrocortisone Acetate, Deoxycorticosterone Acetate, Aldosterone, Beclometasone, Budesonide, Mometasone Furoate, Fluocinolone, Flunisolide, Fluorometholone, Fluticasone, Dagrocorat, Dagrocorat Hydrochloride, Mapracorat, Fosdagrocorat, GSK9027, GSK866, AZD2906, GW-870086, BAY 1003803, ZK 216348, LEO 134310, and RU28362. In some embodiments, the glucocorticoids in step (d) are Hydrocortisone 21-hemisuccinate and Dexamethasone. In some embodiments, Hydrocortisone 21-hemisuccinate is present in the culture medium at a concentration of 10 nM- 100 μM. In some embodiments, Dexamethasone is present in the culture medium at a concentration of 500 pM-5 μM. In some embodiments, the glucocorticoids in step (e) are Hydrocortisone 21-hemisuccinate and Dexamethasone. In some embodiments, Hydrocortisone 21-hemisuccinate is present in the culture medium at a concentration of 10 nM-100 μM. In some embodiments, Dexamethasone is present in the culture medium at a concentration of 500 pM-5 μM.

In some embodiments, the CREB agonist is selected from the group consisting of Forskolin, CAMP, Dibutyryl cAMP, 8-Br-cAMP, Sp-cAMPS, and CW 008. In some embodiments, the CREB agonist is Forskolin. In some embodiments, Forskolin is present in the culture medium at a concentration of 20 nM-2 mM.

In some embodiments, the antioxidant in steps (d) and (e) is independently selected from the group consisting of N-Acetyl-L-Cysteine, Ascorbic Acid, Sodium Ascorbate, Glutathione, Ebselen, α-tocopherol, β-tocopherol, δ-tocopherol, γ-tocopherol, Lipoic Acid, Uric Acid, and Ubiquinol. In some embodiments, the antioxidants in step (d) are N-Acetyl-L-Cysteine, Ascorbic Acid, and Sodium Ascorbate. In some embodiments, N-Acetyl-L-Cysteine is present in the culture medium at a concentration of 500 nM-2 M. In some embodiments, Ascorbic Acid is present in the culture medium at a concentration of 250 pg/ml-25 mg/ml. In some embodiments, Sodium Ascorbate is present in the culture medium at a concentration of 200 pg/ml-20 mg/ml. In some embodiments, the antioxidant in step (e) is Sodium Ascorbate. In some embodiments, Sodium Ascorbate is present in the culture medium at a concentration of 200 pg/ml-20 mg/ml.

In some embodiments, the Wnt antagonist in each of steps (d) and (e) is independently selected from the group consisting of IWR-1-endo, C59, XAV939, WIKI4, JW55, JW74, NVP-TNKS656, LZZ-02, TC-E 5001, IWP2, IWP4, WNT974, CGX1321, ETC-159, RXC004, GNF-6231, WIF-1, Ipafricept, DKK1, BMD4503-2, Salinomycin, NSC 668036, FJ9, 3289-8625, LF3, CCT036477, CCT251545, MSAB, KY1220, KY02111, FH535, Triptonide, KYA1797K, iCRT3, iCRT5, iCRT14, PNU-74654, PKF118-310, Cardionogen 1, ICG-001, JW67, NLS-StAx-h, PRI-724, GNE-781, Capmatinib, NCB-0846, TAK715, Nitazoxanide, Vantictumab, OTSA-101, and Fz7-21. In some embodiments, the Wnt antagonist in step (d) is IWR-1-endo. In some embodiments, IWR-1-endo is present in the culture medium at a concentration of 500 pM- 10 μM. In some embodiments, the Wnt antagonists in step (e) are IWR-1-endo and C59. In some embodiments, IWR-1-endo is present in the culture medium at a concentration of 500 pM-10 μM. In some embodiments, C59 is present in the culture medium at a concentration of 5 nM-100 μM.

In some embodiments, the TGF-β antagonist in each of steps (d) and (e) is independently selected from the group consisting of SB431542, A 83-01, GW788388, SB505124, SB525334, TP0427736, RepSox, SD-208, Galunisertib, IN-1130, Ki 26894, LY2109761, LY2157299, LY550410, PF-03446962, TEW-7197, AP12009, AP11014, AP15012, ISTH0036, Fresolimumab, Lerdelimumab, GC1008, 2G7, 1D11, CAT-192, LY2382770, and LY3022859. In some embodiments, the TGF-β antagonist in step (d) is SB431542. In some embodiments, SB431542 is present in the culture medium at a concentration of 10 nM-200 μM. In some embodiments, the TGF-β antagonist in step (e) is SB431542. In some embodiments, SB431542 is present in the culture medium at a concentration of 10 nM-200 μM.

In some embodiments, the HGF agonist in each of steps (d) and (e) is independently selected from the group consisting of HGF, Dihexa, NK1, NK2, Fosgonimeton, and Terevalefim. In some embodiments, the HGF agonists in step (d) are HGF and Dihexa. In some embodiments, HGF is present in the culture medium at a concentration of 100 pg/ml-10 μg/ml. In some embodiments, Dihexa is present in the culture medium at a concentration of 500 pg/ml-1 μg/ml. In some embodiments, the HGF agonist in step (e) is Dihexa. In some embodiments, Dihexa is present in the culture medium at a concentration of 500 pg/ml-1 μg/ml.

In some embodiments, the Notch antagonist is selected from the group consisting of DAPT, GSI-XX, BMS 299897, BMS 433796, BMS 906024, BMS 986115, Compound E, Compound W, Compound 18, DBZ, DFK-167, L-685458, LY 3039478, LY 411575, LY 450139, LY 900009, MK-0752, MRK 003, MRK 560, PF 3084014, PF 3084014 Hydrobromide, Z-IL-CHO, Avagacestat, Begacestat, JLK6, AL101, RO 4929097, FLI-06, Thapsigargin, CAD204520, Tangeretin, Bruceine D, 15D11, Enoticumab, Demcizumab, ABT-165, Navicixizumab, Marimastat, ZLDI-8, IMR-1, IMR-1A, CB-103, RIN1, Brontictuzumab, Tarextumab, and PF 06650808. In some embodiments, the Notch antagonist is DAPT. In some embodiments, DAPT is present in the culture medium at a concentration of 10 nM- 100 μM.

In some embodiments, the BMP antagonist is selected from the group consisting of LDN193189, Dorsomorphin, DMH-1, DMH-2, ML347, LDN212854, LDN214117, K02288, Follistatin, Follistatin-like 1, Noggin, Chordin, Ventroptin, Twisted Gastrulation, Dan, Cerberus, PRDC, Dante, Caronte, Gremlin, and Sclerostin. In some embodiments, the BMP antagonist is LDN193189. In some embodiments, LDN193189 is present in the culture medium at a concentration of 500 pM-2.5 μM.

In some embodiments, the hepatocyte function enhancer is FH1 or FPH1. In some embodiments, the hepatocyte function enhancer is FH1. In some embodiments, FH1 is present in the culture medium at a concentration of 10 nM-2.5 mM.

In another aspect, the disclosure pertains to various culture media for obtaining endodermal cells, midgut cells, hepatoblasts, or hepatocytes. In some embodiments, the disclosure provides a culture medium for obtaining endodermal cells comprising a GSK3β inhibitor. In some embodiments, the disclosure provides a culture medium for obtaining midgut cells comprising an HDAC inhibitor, a RA agonist, and an FGFR (e.g., FGFR1) agonist. In some embodiments, the disclosure provides a culture medium for obtaining hepatoblasts comprising a glucocorticoid, a CREB agonist, an antioxidant, a Wnt antagonist, a TGF-β antagonist, and an HGF agonist. In some embodiments, the disclosure provides a culture medium for obtaining hepatocytes comprising an HDAC inhibitor, a glucocorticoid, a CREB agonist, an antioxidant, a Wnt antagonist, a TGF-β antagonist, an HGF agonist, a Notch antagonist, a BMP antagonist, and a hepatocyte function enhancer.

In another aspect, the disclosure pertains to isolated cell cultures of endodermal cells, midgut cells, hepatoblasts, or hepatocytes. In some embodiments, the disclosure provides an isolated cell culture comprising endodermal cells in a culture medium comprising a GSK36 inhibitor. In some embodiments, the disclosure provides an isolated cell culture comprising midgut cells in a culture medium comprising an HDAC inhibitor, a RA agonist, and an FGFR (e.g., FGFR1) agonist. In some embodiments, the disclosure provides an isolated cell culture comprising hepatoblasts in a culture medium comprising a glucocorticoid, a CREB agonist, an antioxidant, a Wnt antagonist, a TGF-β antagonist, and an HGF agonist. In some embodiments, the disclosure provides an isolated cell culture comprising 50 million to 50 billion hepatocytes in a culture medium comprising an HDAC inhibitor, a glucocorticoid, a CREB agonist, an antioxidant, a Wnt antagonist, a TGF-β antagonist, an HGF agonist, a Notch antagonist, a BMP antagonist, and a hepatocyte function enhancer.

In another aspect, the disclosure pertains to endodermal cells, midgut cells, hepatoblasts, or hepatocytes generated by the methods of the disclosure. In some embodiments, the disclosure provides endodermal cells generated by a method of the disclosure. In some embodiments, the disclosure provides midgut cells generated by a method of the disclosure. In some embodiments, the disclosure provides hepatoblasts generated by a method of the disclosure. In some embodiments, the disclosure provides hepatocytes generated by a method of the disclosure.

In yet another aspect, the disclosure provides an isolated cell culture comprising 50 million to 50 billion engineered hepatocytes generated by a method of the disclosure.

Other features and advantages of the invention will be apparent from the following detailed description and claims.

### BRIEF DESCRIPTION OF THE DRAWINGS

FIG. 1 is a schematic showing the protocol for producing iPSC-derived hepatocytes. Components and concentrations of reagents used at the different stages of the process are all under the appropriate protocol stage. Protocol was defined through High-Dimensional Design of Experiments (HD-DoE) modelling experiments.

FIG. 2 is a schematic of the approach taken for HD-DoE informed differentiation optimization strategy. The genes focused on in the discrete stage of protocol development are listed below the individual stages. All target genes were optimized for maximal expression, with the exception of AFP in stage 4. AFP was minimized in stage 4.

FIG. 3A is a schematic showing the outline for HD-DoE-informed optimization for SOX17 expression.

FIG. 3B shows the experimental results for the optimal induction of SOX17.

FIG. 4A is a schematic showing the outline for HD-DoE-informed optimization for FOXA2 and HNF1B expression.

FIG. 4B shows the experimental results for the optimal induction of FOXA2 and HNF1B.

FIG. 4C shows the comparison of genes induced through incubation with IDE1, IDE2, and activin A.

FIG. 5A is a schematic showing the outline for HD-DoE-informed optimization for HNF1B expression.

FIG. 5B shows the experimental results for the optimal induction of HNF1B.

FIG. 6 is a heatmap showing the factors that drive endoderm induction. All endoderm markers measured throughout this experiment were optimized for maximal expression. The genes being optimized are listed at the top and the differentiation-inducing reagents are listed on the left side of the heatmap. The impact of the effectors being evaluated on overall gene induction are listed as factor contribution (FC) numbers. Larger FC numbers are indicative of the overall strength of the effect. Positive FC numbers represent positive effects and negative FC numbers indicate negative effects on the induction of the gene being investigated. The overall effects of the genes measured were added across all genes investigated and the average sum of the effects are listed on the right side of the heatmap.

FIGS. 7A-7D are graphs showing the evaluation of opposing endoderm induction methods for generating an improved hepatic field. Two basic methods used for endoderm induction are present in the literature: 1) 3-day induction using AA/WNT3a and 2) 1-day induction using CHIR. Different methods were directly compared to a C/D/H/I induction developed using HD-DoE modeling. The timing of the induction was included in design to account for the different timing of the two literature approaches. FIGS. 7A and 7C show the predicted levels of the different genes over a 3-day differentiation event. To evaluate the commonly used endoderm inductions methods in silico models removing all effectors except for the different conditions shown stated in the figure legends. FIGS. 7B and 7D show using an in silico approach, the overall effects of the individual component for the C/D/H/I approach were evaluated models that removed each of the small molecules independently. These secondary models are provided beneath graphs evaluating the different approaches. Positive slope in these models indicates a contribution for inducing the gene being investigated.

FIG. 8A is a schematic showing the outline for HD-DoE-informed optimization for SOX17, FOXA2, and HFN1B expression.

FIGS. 8B-8D show the experimental results for the optimal induction of SOX17, FOXA2, and HNF1B, respectively.

FIG. 9 shows a dynamic profile evaluation confirming that Activin A, DMSO, and CHIR play a role in inducing the core endoderm genes. No synergy between these compounds was shown, suggesting that the effects of the compounds were independent of each other.

FIG. 10A is a schematic showing the outline for HD-DoE-informed optimization for SOX17, FOXA2, and HNF1B co-expression.

FIG. 10B shows the experimental results for the optimal induction of co-expression of SOX17, FOXA2, and HNF1B.

FIG. 11A is a schematic showing the differentiation experiment for generation of endodermal cells.

FIG. 11B shows IHC images demonstrating the co-expression of SOX17 and FOXA2, a hallmark of endoderm induction.

FIG. 12A is a schematic showing the differentiation experiment for generation of endodermal cells.

FIG. 12B shows IHC imaging demonstrating the expression patterns for the endoderm specific gene SOX17 and early hepatic genes NR5A2, HNF4A, PROX1, and ALB.

FIG. 13 is a schematic showing normal embryonic endoderm expression patterns along the gut tube when initially formed and before organogenesis begins. The midgut region, where the liver bud forms, is well defined through the co-expression of HHEX, ONECUT1, and EVX1.

FIG. 14A is a schematic showing the outline for HD-DoE-informed optimization for HHEX, EVX1,and PROX1 expression.

FIGS. 14B-14D show the experimental results for the optimal induction of HHEX, EVX1, and PROX1, respectively.

FIG. 15A is a schematic showing the outline for HD-DoE-informed optimization for PROX1 expression.

FIG. 15B shows the experimental results for the optimal induction of PROX1.

FIG. 16A is a heatmap showing the FC values of the genes that represent the different descendent lineages of the liver.

FIG. 16B shows the average contribution factors for the different effectors tested.

FIG. 17A is a schematic showing the outline for HD-DoE-informed optimization for HHEX, EVX1, and ONECUT1 expression.

FIGS. 17B-17D show the experimental results for the optimal induction of HHEX, EVX1, and ONECUT1, respectively.

FIG. 18A is a heatmap showing the FC values of the genes that represent the different descendent lineages of the liver.

FIG. 18B shows the average contribution factors for the different effectors tested.

FIG. 19 is a heatmap showing the factors that drive a hepatic fate from endoderm. Key endoderm genes were modeled for maximal induction from three sequential modeling experiments that evaluated 12 factors at a time. The effectors evaluated are listed in the left column of the heatmap. Genes being interrogated are listed at the top of the heatmap. The impact of the effectors on gene induction is listed as FC numbers. Larger FC numbers are indicative of the overall effector strength on the induction of the gene. Positive FC numbers represent positive inducing effects and negative FC numbers have inhibitory effects on the induction of the gene being investigated.

FIG. 20A is a schematic of the differentiation experiment for generation of midgut cells.

FIG. 20B shows IHC images demonstrating that SOX17, a pan-endodermal marker, was widely expressed in the descendent iPSC-derived midgut population. The more hepatic specific genes from this region, AFP and HNF4A, were also shown to be induced in this culture.

FIG. 21A is a schematic of the differentiation experiment for generation of midgut cells.

FIG. 21B shows IHC images demonstrating that when working models for midgut induction were tested empirically by incubating endoderm culture in the presence of Vorinostat, TTNPB, and FGF2 for five-days, TBX3 expression is induced.

FIG. 22A is a schematic of the differentiation experiment for generation of immature hepatocytes.

FIG. 22B shows IHC images validating that midgut generated with Vorinostat, TTNPB, and FGF2 is competent to differentiate towards a hepatic fate.

FIG. 23A is a schematic of the differentiation experiment for generation of immature hepatocytes.

FIG. 23B shows IHC images demonstrating expression patterns of AFP, ALB, and HNF4A in the hepatoblasts.

FIG. 24 is a schematic showing genes that are differentially expressed throughout the development of different liver-specific fates including the early hepatoblast progenitor, cholangiocytes (ductal cells), and hepatocytes with both an immature and a mature phenotype.

FIG. 25A is a schematic showing the outline for HD-DoE-informed optimization for HNF4A and AFP expression.

FIG. 25B shows the experimental results for the optimal induction of HNF4A and AFP.

FIG. 26A is a schematic showing the outline for HD-DoE-informed optimization for HNF4A, TBX3, CYP3A7, and AFP expression.

FIGS. 26B-26E show the experimental results for the optimal induction of HNF4A, TBX3, CYP3A7, and AFP, respectively.

FIG. 27A is a schematic showing the outline for HD-DoE-informed optimization for HNF4A, AFP, and TBX3 expression.

FIGS. 27B-27D show the experimental results for the optimal induction of HNF4A, AFP, and TBX3, respectively.

FIG. 28A is a schematic showing the outline for HD-DoE-informed optimization for AFP and CYP3A7 expression.

FIG. 28B shows the experimental results for the optimal induction of AFP and CYP3A7.

FIG. 29A is a schematic showing the outline for HD-DoE-informed optimization for HNF4A and AFP.

FIG. 29B shows the experimental results for the optimal induction of HNF4A and AFP.

FIG. 30A is a heatmap showing the factors that drive a hepatic field. An HD-DoE approach was used to evaluate how to optimally pattern a culture representative of a hepatoblast from the stage 2 midgut cultures. Five sequential modeling experiments evaluating 12 factors at a time were tested in this approach. Factors that were evaluated are listed in the left column of the heat map. All of the genes listed at the top were optimized for maximum expression within MODDE software and the differentiation-inducing reagents'impact on the gene induction is listed as FC numbers. Larger FC numbers are indicative of the overall strength of the effect, while positive FC numbers represent positive-inducing effects and negative FC numbers represent negative (inhibitory) effects on the induction of the gene being investigated.

FIG. 30B shows the sum of the FC values for all the genes interrogated in this manner.

FIG. 31A is a schematic of the differentiation experiment for generation of hepatocytes.

FIG. 31B shows IHC images of Albumin, A1AT, and Bodipy staining, which indicate lipid drop retention.

FIG. 32A is a schematic of the differentiation experiment for generation of hepatocytes.

FIG. 32B shows IHC images demonstrating the expression of TBX3, ZO1, and CYP3A4 in both iPSC-derived hepatocytes and primary hepatocytes.

FIG. 33A shows the level of albumin secreted over a four-day time course when bioreactor-generated iPSC-derived hepatocytes were plated onto three different ECM molecules and evaluated for albumin secretion.

FIG. 33B shows IHC images demonstrating the expression of AFP and ALB on the three ECM molecules evaluated.

FIG. 33C shows results from RNA analysis of the expression levels of ALB and AFP on the three different substrates. No statistical difference in albumin expression or secretion was detected on any of the different ECM substrates tested. However, a statistically significant decrease in AFP expression could be achieved depending on what ECM molecules are present.

FIG. 34A demonstrates that further evidence of lipid drop accumulation can be obtained from close examination of brightfield images of the iPSC-derived hepatocytes.

FIG. 34B shows that lipid drops are clearly visible (indicated using arrows).

FIGS. 34C-34E show widespread accumulation of the Bodipy dye within the iPSC-derivatives.

FIG. 35 shows IHC images demonstrating clear evidence of cells containing multiple nuclei.

FIG. 36A is a schematic of the differentiation experiment for generation of hepatocytes.

FIG. 36B shows IHC images demonstrating that iPSC-derived hepatocytes contain different numbers of nuclei per cell.

FIG. 37A is a schematic of the differentiation experiment for generation of hepatocytes.

FIG. 37B shows IHC images demonstrating the expression patterns of AFP and CK18 in iPSC-derived hepatocytes. There is clear visible evidence of canaliculi formation within the culture. The white dotted line outlines canaliculus areas.

FIG. 38 shows images demonstrating the transport of CDF (middle row) from iPSC-derived hepatocytes to pseudo-canaliculus area.

FIG. 39A is a schematic showing the outline for HD-DoE-informed optimization for CYP3A4, ALB, TF, and CYP2C9 expression.

FIGS. 39B-39E show experimental results for the optimal induction of CYP3A4, ALB, TF (Transferrin), and CYP2C9, respectively.

FIG. 40A is a schematic showing the outline for HD-DoE-informed optimization for CYP3A4, ALB, TF, and CY2C9 expression.

FIGS. 40B-40E show experimental results for the optimal induction of CYP3A4, ALB, TF (Transferrin), and CYP2C9, respectively.

FIG. 41 is a heatmap of the genes involved in additional HD-DoE modeling experiments that were aimed at increasing the functional level of the iPSC-derived hepatocytes. All of the genes measured that are associated with mature hepatocytes were optimized individually and the representative contribution factors for the components evaluated are shown.

FIG. 42 shows images demonstrating formation of canaliculi between two or multiple adjacent cells and the production of CDF (middle row).

FIG. 43A is a schematic of the experiment performed to visualize expression of maturation markers.

FIG. 43B shows IHC images demonstrating the expression patterns of CYP3A4 and MRP2, which is an apical hepatocyte marker that is present within canaliculi.

FIG. 44A is a schematic of the experiment performed to visualize CYP450 activity.

FIG. 44B shows measurement of CYP activities in iPSC-derived hepatocytes in the presence of a colorimetric substrate for CYP2C9.

FIG. 45A is a schematic of the experiment performed to visualize CYP3A4 activity with and without a CYP3A4 inhibitor.

FIG. 45B shows measurement of CYP activities in iPSC-derived hepatocytes in the presence of a colorimetric substrate for CYP2C9 and the presence of specific CYP3A4 inhibitor (ketoconazole). All reactions were performed on cryopreserved and recovered cells.

FIG. 46 is a set of bar graphs showing RNA-based evaluation of the transition from stage 3 to stage 4 cultures for the relative expression of TF, TBX3, A1AT and HNF4A. iPSC-derived hepatocytes were directly compared to primary hepatocytes.

FIG. 47 is a set of bar graphs showing stage-wise assessment of hepatocyte induction protocol. The expression patterns of the iPSC-derived hepatocytes produced through this series of experiments were directly compared to primary hepatocytes. It was shown that there were no significant differences in the level of expression for the genes assayed in this figure.

FIG. 48 is a set of bar graphs showing stage-wise assessment of hepatocyte induction protocol. The expression patterns of the iPSC-derived hepatocytes produced through this series of experiments were directly compared to primary hepatocytes. It was shown to have minimal significant difference in the level of expression for the genes assayed in this figure.

FIG. 49 is a heatmap showing changes in the genes in various cell populations obtained throughout the differentiation protocol. Liver-specific genes are upregulated in iPSC-derived hepatocytes.

FIG. 50 demonstrates density-dependent expression of albumin in iPSC-derived hepatocytes.

FIG. 51 demonstrates density-dependent expression of CYP3A4 and UGT1A1 in iPSC-derived hepatocytes.

### DETAILED DESCRIPTION OF THE INVENTION

Described herein are methodologies and compositions that allow for the generation of hepatocytes from induced pluripotent stem cells (iPSCs) under chemically-defined culture conditions using a small molecule based approach. The methods of the disclosure generate hepatocytes in a five-step, four-stage protocol in which endodermal cells are generated in 1 day, followed by generation of endodermal cells by day 2 of culture, followed by generation of midgut cells by day 7 of culture, followed by generation of hepatoblasts by day 17 of culture, followed by generation of hepatocytes by day 24 of culture. Thus, the disclosure allows for obtention of hepatocytes in a significantly shorter time than prior art protocols using chemically-defined culture conditions.

High-Dimensional Design of Experiments (HD-DoE) approach was used to develop the methods of the disclosure. The HD-DoE approach starts with iPSCs, builds up a protocol through high dimensional testing, applies a Quality-by-Design schema, and implements DoE/MVDA iteratively for each stage. As a result, the resulting protocol demonstrates multiparameter control of cell-fate and consistently demonstrates high purity and functionality of the descendant cell-types. Specifically, the HD-DoE approach simultaneously tests multiple process inputs (e.g., small molecule agonists or antagonists) on output responses, such as gene expression. This approach allowed for the identification of chemically-defined culture media, comprising agonists or antagonists of particular signaling pathways, that is sufficient to generate endodermal cells, midgut cells, hepatoblasts, and hepatocytes under defined conditions in a short amount of time. The optimized culture media was further validated by a factor criticality analysis, which examined the effects of eliminating individual agonist or antagonist agents. Various aspects of the invention are described in further detail in the following subsections.

### I. Cells

The starting cells used in the cultures of the disclosure are induced pluripotent stem cells (iPSCs). As used herein, the terms “induced pluripotent stem cell” and “iPSC” refer to a cell taken from a later point in development that has been induced to have expression patterns consistent with a pluripotent cell. The source of the cell can be either embryonic or adult in origin. Non-limiting examples of iPSCs are CR01, 19-11-1, 19-9-7, and 6-8-8 cells (e.g., as described in Yu, J. et al. (2009) Science 324:797-801).

Human pluripotent stem cells (PSCs) express cellular markers that can be used to identify cells as being PSCs. Non-limiting examples of PSC markers include TRA-1-60, TRA-1-81, TRA-2-54, SSEA1, SSEA3, SSEA4, CD9, CD24, OCT3, OCT4, NANOG, and SOX2. Since the methods of generating endodermal cells of the disclosure are used to differentiate (maturate) the starting iPSC population, the endodermal cells generated by the methods of the disclosure lack expression of one or more stem cell markers, such as one or more stem cell markers selected from the group consisting of TRA-1-60, TRA-1-81, TRA-2-54, SSEA1, SSEA3, SSEA4, CD9, CD24, OCT3, OCT4, NANOG, and SOX2.

iPSCs are subjected to culture conditions, as described herein, that induce cellular differentiation. As used herein, the term “differentiation” refers to the development of a cell from a more primitive stage towards a more mature (i.e. less primitive) cell, typically exhibiting phenotypic features of commitment to a particular cellular lineage.

In embodiments, cells can be identified and characterized based on expression of one or more biomarkers, such as particular biomarkers of hepatocyte-committed PSCs. A “positive” biomarker is one that is expressed on a cell of interest, whereas a “negative” biomarker is one that is not expressed on a cell of interest.

Non-limiting examples of biomarkers whose expression can be evaluated in the characterization of endodermal cells generated by the methods of the disclosure are SOX17, FOXA2, and HNF1B.

Non-limiting examples of biomarkers whose expression can be evaluated in the characterization of midgut cells generated by the methods of the disclosure are NR5A2, HNF6, HHEX, EVX1, PROX1,and TBX3.

Non-limiting examples of biomarkers whose expression can be evaluated in the characterization of hepatoblasts generated by the methods of the disclosure are AFP, ALB, A1AT, ATF5, FAH, TF, CEBPA, TBX3, HNF4A, AHSG, CD99, APOA1, and APOA2.

Non-limiting examples of biomarkers whose expression can be evaluated in the characterization of hepatocytes generated by the methods of the disclosure are DLK1, GPC3, CYP3A7, FXR, CAR, PXR, ASS, ARG1, FGL1, ORM1, CYP2C9, and CYP3A4.

As used herein, expression by a cell of only “low” levels of a biomarker of interest is intended to refer to a level that is at most 20%, and more preferably, less than 20%, less than 15%, less than 10%, or less than 5% above background levels (wherein background levels correspond to, for example, the level of expression of a negative control marker that is considered to not be expressed by the cell).

In some embodiments, the cells generated by the methods of the disclosure are endodermal cells. As used herein, the term “endodermal cell” refers to a cell that expresses at least one biomarker, and preferably two or all three biomarkers selected from SOX17, FOXA2, and HNF1B.

In some embodiments, the cells generated by the methods of the disclosure are midgut cells. As used herein, the term “midgut cell” refers to a cell that expresses at least one biomarker, and preferably two or more biomarkers selected from NR5A2, HNF6, HHEX, EVX1, PROX1, and TBX3.

In some embodiments, the cells generated by the methods of the disclosure are hepatoblasts. As used herein, the term “hepatoblast” refers to a cell that expresses at least one biomarker, and preferably two or more biomarkers selected from AFP, ALB, A1AT, ATF5, FAH, TF, CEBPA, TBX3, HNF4A, AHSG, CD99, APOA1, and APOA2.

In some embodiments, the cells generated by the methods of the disclosure are hepatocytes. As used herein, the term “hepatocyte” refers to a cell that expresses at least one biomarker, and preferably two or all biomarkers selected from DLK1, GPC3, and CYP3A7 where not fully mature, and FXR, CAR, PXR, ASS, ARG1, FGL1, ORM1, CYP2C9, and CYP3A4 where fully mature. A hepatocyte may also express additional biomarkers, including but not limited to, AFP, ALB, A1AT, ATF5, FAH, and TF.

As used herein, the term “engineered” cells refers to cells that are not naturally-occurring. Engineered cells may result from genetic engineering, which modifies cells to contain and/or express a foreign gene or nucleic acid sequence, which, in turn, modifies the genotype and/or phenotype of the cells or their progeny. Engineered cells may also be obtained from preconditioning, biomaterial encapsulation, surface modification, or cell assembly approaches. For stem cells, engineered cells may be obtained from a directed differentiation process that involves external factors.

### II. Culture Media Components

The methods of the disclosure for generating endodermal cells, midgut cells, hepatoblasts, and hepatocytes from iPSCs comprise culturing iPSCs in culture media comprising specific agonists and/or antagonists of cellular signaling pathways.

A culture media comprising a GSK30 inhibitor was sufficient to generate endodermal cells in as little as two days (referred to herein as “Stage 1” of the differentiation protocol). Further differentiation of the endodermal cells in a culture medium comprising an HDAC inhibitor, a RA agonist, and an FGFR (e.g., FGFR1) agonist was sufficient to generate midgut cells in another five days (referred to herein as “Stage 2”). Further differentiation of the midgut cells in a culture medium comprising a glucocorticoid, a CREB agonist, an antioxidant, a Wnt antagonist, a TGF-β antagonist, and an HGF agonist was sufficient to generate hepatoblasts in another 10 days (referred to herein as “Stage 3”). Still further differentiation of the hepatoblasts in a culture medium comprising a glucocorticoid, an HGF agonist, an antioxidant, a TGF-β antagonist, a CREB agonist, a Notch antagonist, a BMP antagonist, a Wnt antagonist, an HDAC inhibitor, and a hepatocyte function enhancer was sufficient to generate hepatocytes in another seven days (referred to herein as “Stage 4”), for an overall four-stage, 24-day protocol for generating hepatocytes.

As used herein, the term “agonist” refers to an agent that stimulates a cellular signaling pathway. Stimulation of the cellular signaling pathway can be initiated extracellularly, for example, by use of an agonist that activates a cell surface receptor involved in the signaling pathway (e.g., the agonist can be a receptor ligand). Additionally or alternatively, stimulation of the cellular signaling pathway can be initiated intracellularly, for example, by use of a small molecule agonist that interacts intracellularly with one or more components of the signaling pathway.

As used herein, the term “antagonist” refers to an agent that inhibits a cellular signaling pathway. Inhibition of the cellular signaling pathway can be initiated extracellularly, for example, by use of an antagonist that blocks a cell surface receptor involved in the signaling pathway. Additionally or alternatively, inhibition of the cellular signaling pathway can be initiated intracellularly, for example, by use of a small molecule antagonist that interacts intracellularly with one or more components of the signaling pathway.

Agonists and antagonists used in the methods of the disclosure are known in the art and commercially available. They are used in the culture media at a concentration effective to achieve the desired outcome, e.g., generation of hepatocytes expressing hepatocyte-associated biomarkers. Non-limiting examples of suitable agonistic and antagonistic agents and effective concentration ranges are described further below.

As used herein, the terms “glycogen synthase kinase 3 beta inhibitor” and “GSK3β inhibitor” refer to an agent that inhibits or decreases signaling through or activity of a GSK3β enzyme. In some examples, a GSK3β inhibitor is capable of (i) decreasing phosphorylation of GSK3B, (ii) decreasing phosphorylation of β-catenin, (iii) increasing expression of β-catenin, (iv) decreasing phosphorylation of PTEN, (v) decreasing phosphorylation of mTOR, or (vi) decreasing phosphorylation of AKT. In some embodiments, the GSK36 inhibitor binds a GSK36 enzyme and inhibits or decreases signaling through or activity of the GSK36 enzyme. In some embodiments, the GSK30 inhibitor is ATP-competitive. In some embodiments, the ATP-competitive GSK3B inhibitor is CHIR99021, CHIR98014, CHIR98023, 3F8, A1070722, AR-A014418, BIO, BIO-acetoxime, 6-BIO, Indirubin-3′-oxime, Alsterpaullone, 1-Azakenpaullone, Cazpaullone, Kenpaullone, Aloisine A, SB216763, SB415286, SB41528, SAR502250, TC-G 24, TWS119, LY2090314, AT7519, KY19382, AZD1080, AZD2858, Hymenialdesine, Debromohymenialdisine, Dibromocantherelline, Meridianine A, NSC 693868, IM-12, IMID1, IMID2, VP2.51, VP2.54, BIP-135, JGK-263, MMBO, TCS2002, PF-367, BRD0705, BRD3731, or AF3581. In some embodiments, the GSK36 inhibitor is not ATP-competitive. In some embodiment, s the non-ATP-competitive GSK3β inhibitor is TDZD-8, NP00111, Tideglusib (NP031112), NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, or 4-4.

In some embodiments, the GSK33 inhibitor is selected from the group consisting of CHIR99021, CHIR98014, CHIR98023, 3F8, A 1070722, AR-A014418, BIO, BIO-acetoxime, 6-BIO, Indirubin-3′-oxime, Alsterpaullone, 1-Azakenpaullone, Cazpaullone, Kenpaullone, Aloisine A, SB 216763, SB 415286, SB41528, SAR502250, TC-G 24, TWS119, LY2090314, AT7519, KY19382, AZD1080, AZD 2858, Hymenialdisine, Debromohymenialdisine, Dibromocantherelline, Meridianine A, NSC 693868, IM-12, IMID1, IMID2, VP2.51, VP2.54, BIP-135, JGK-263, MMBO, TCS2002, PF-367, BRD0705, BRD3731, AF3581, TDZD-8, NP00111, Tideglusib (NP031112), NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, and 4-4. In one embodiment, the GSK3β inhibitor is CHIR99021. In one embodiment, the GSK3B inhibitor is CHIR99021, which is present in the culture medium at a concentration within a range of 100 nM-10 μM. In one embodiment, the GSK3β inhibitor in step (a) (Stage 1) is CHIR99021, which is present in the culture medium at a concentration of 3 μM.

As used herein, the terms “histone deacetylase inhibitor” and “HDAC inhibitor” refer to an agent that inhibits or decreases signaling through or activity of one or more HDAC isoforms (e.g., Class I, which includes HDAC1, HDAC2, HDAC3, and HDAC8; Class IIa, which includes HDAC4, HDAC5, HDAC7, and HDAC9; Class IIb, which includes HDAC6 and HDAC10; Class III, which includes SIRT1-7; Class IV, which includes HDAC11). In some examples, an HDAC inhibitor is capable of decreasing histone acetylation. In some embodiments, the HDAC inhibitor binds one or more HDAC isoforms and inhibits or decreases signaling through or activity of the one or more HDAC isoforms. In some embodiments, the HDAC inhibitor is a pan-HDAC inhibitor. In some embodiments, the HDAC inhibitor is an isoform-selective inhibitor.

In some embodiments, the HDAC inhibitor is selected from the group consisting of Vorinostat, Sodium Butyrate, Belinostat, Dacinostat, Droxinostat, Domatinostat, Entinostat, Ricolinostat, Resminostat, Abexinostat, Givinostat, Ivaltinostat, Panobinostat, Pracinostat, Quisinostat, Mocetinostat, Nanatinostat, Nexturastat A, Tefinostat, Tucidinostat, Fimepinostat, Citarinostat, Zabadinostat, GSK3117391, CUDC-101, AR-42, M344, Scriptaid, Sulforaphane, SR-4370, MC1568, CAY10603, CAY10683, RG2833, RGFP966, Cpd60, BRD3308, Tasquinimod, BML-210, LMK-235, BRD73954, PCI-34051, TMP195, TMP269, NKL22, TH34, SIS17, WT-161, ACY-738, BG45, CBHA, Pyroxamide, NCH-51, NCH-31, KD 5170, TCS HDAC6 20b, NSC 3852, NSC 69603, NSC 86371, NSC 305819, MS-27-275, Trapoxin A, Trapoxin B, Romidepsin, Apicidin, Trichostatin A, ACY-775, Tubastatin A, Tubacin, SKLB-23bb, HPOB, Curcumin, UF010, tc-H 106, Splitomicin, Raddeanin A, Depudecin, Tacedinaline, Isoguanosine, Parthenolide, Tinostamustine, Sodium Phenylbutyrate, Valproic Acid, Butyric acid, Phenylbutyric acid, 4-Phenylbutyric Acid, Divalproex Sodium, Sinapinic Acid, Suberohydroxamic Acid, Biphenyl-4-sulfonyl chloride, Thiophene Benzamide, Nicotinamide, Dihydrocoumarin, Naphthopyranone, and 2-Hydroxynaphthaldehyde. In one embodiment, the HDAC inhibitor is Vorinostat. In one embodiment, the HDAC inhibitor is Vorinostat, which is present in the culture medium at a concentration within a range of 10 nM-5 μM. In one embodiment, the HDAC inhibitor in step (c) (Stage 2) is Vorinostat, which is present in the culture medium at a concentration of 100 nM. In one embodiment, the HDAC inhibitor is Sodium Butyrate. In one embodiment, the HDAC inhibitor is Sodium Butyrate, which is present in the culture medium at a concentration within a range of 10 nM-5 μM. In one embodiment, the HDAC inhibitor in step (e) (Stage 4) is Sodium Butyrate, which is present in the culture medium at a concentration of 250 nM.

As used herein, the terms “retinoic acid agonist” and “RA agonist” refer to an agent that stimulates or increases signaling through or activity of one or more retinoic acid receptor (RAR) subtypes (e.g., RARα, RARβ, and RARγ). In some examples, a RA agonist is capable of increasing RAR mRNA levels. In some embodiments, the RA agonist binds one or more RAR subtypes and stimulates or increases signaling through or activity of the one or more RAR subtypes. In some embodiments, the RA agonist is a pan-RAR agonist. In some embodiments, the pan-RAR agonist is TTNPB, ATRA, 9-cis-Retinoic Acid, Adapalene, Tretinoin, WYC-209, DC271, Acitretin, Arotinoid, AGN190168, AGN205327, or LGD1550. In some embodiments, the RA agonist is a RARα/βagonist. In some embodiments, the RARα/βagonist is Ch55. In some embodiments, the RA agonist is selective for RARα. In some embodiments, the RARα-selective agonist is AM580 (CD336), CD2081, BMS 753, Tamibarotene, AGN194078, AGN195183, or AGN193836. In some embodiments, the RA agonist is selective for RARβ. In some embodiments, the RARβ-selective agonist is CD2314, CD2019, CD666, C286, BMS 641, AC-55649, AC261066, or KCL-286. In some embodiments, the RA agonist is selective for RARγ. In some embodiments, the RARγ-selective agonist is CD1530, CD437, CD2325, BMS 189961, BMS 270394, BMS 961, Trifarotene, or Palovarotene.

In some embodiments, the RA agonist is selected from the group consisting of TTNPB, Ch55, CD1530, CD2314, CD437, CD666, CD2019, CD2081, CD2325, BMS 753, BMS 961, BMS 189961, BMS 270394, AM 580, Adapalene, AC-55649, AC-261066, Tamibarotene, ATRA, Tretinoin, and Palovarotene. In one embodiment, the RA agonist is TTNPB. In one embodiment, the RA agonist is TTNPB, which is present in the culture medium at a concentration within a range of 5 nM-2 μM. In some embodiments, the RA agonist in step (c) (Stage 2) is TTNPB, which is present in the culture medium at a concentration of 100 nM.

As used herein, the terms “fibroblast growth factor receptor agonist” and “FGFR (e.g., FGFR1) agonist” refer to an agent that stimulates or increases signaling through or activity of an FGFR (e.g., FGFR1, FGFR2, FGFR3, and FGFR4). In some examples, an FGFR (e.g., FGFR1) agonist is capable of (i) increasing phosphorylation of FRS2a, (ii) increasing phosphorylation of ERK, or (iii) increasing phosphorylation of AKT. In some embodiments, the FGFR (e.g., FGFR1) agonist binds an FGFR and stimulates or increases signaling through or activity of the FGFR. In some embodiments, the FGFR (e.g., FGFR1) agonist is a naturally occurring ligand. In some embodiments, the naturally occurring ligand of FGFR is FGF2, FGF1, FGF3, FGF4, FGF5, FGF6, FGF8, FGF10, FGF17, FGF19, FGF20, FGF21, FGF22, or FGF23. In some embodiments, the FGFR (e.g., FGFR1) agonist is a ligand mimetic. In some embodiments, the FGFR ligand mimetic is SUN11602.

In some embodiments, the FGFR (e.g., FGFR1) agonist is selected from the group consisting of FGF2, SUN11602, FGF1, FGF3, FGF4, FGF5, FGF6, FGF8, FGF10, FGF17, FGF19, FGF20, FGF21, FGF22, and FGF23. In some embodiments, the FGFR (e.g., FGFR1) agonist is FGF2 or SUN11602. In one embodiment, the FGFR (e.g., FGFR1) agonist is FGF2. In one embodiment, the FGFR (e.g., FGFR1) agonist is FGF2, which is present in the culture medium at a concentration within a range of 500 pg/ml-500 ng/ml. In one embodiment, the FGFR (e.g., FGFR1) agonist in step (c) (Stage 2) is FGF2, which is present in the culture medium at a concentration of 10 ng/ml.

As used herein, the term “glucocorticoid” refers to an agent that stimulates or increases signaling through or activity of a glucocorticoid receptor. In some examples, a glucocorticoid is capable of (i) increasing expression of anti-inflammatory proteins in the nucleus, (ii) decreasing expression of pro-inflammatory proteins in the cytosol, or (iii) increasing gluconeogenesis. In some embodiments, the glucocorticoid binds a glucocorticoid receptor and stimulates or increases signaling through or activity of the glucocorticoid receptor. In some embodiments, the glucocorticoid is selected from the group consisting of Hydrocortisone 21-hemisuccinate, Dexamethasone, Dexamethasone Acetate, Hydrocortisone, Cortisone, Prednisone, Prednisone Acetate, Meprednisone, Prednisolone, Methylprednisolone, Methylprednisolone Acetate, Fluprednisolone, Betamethasone, Paramethasone, Triamcinolone, Deflazacort, Fludrocortisone, Fludrocortisone Acetate, Deoxycorticosterone Acetate, Aldosterone, Beclometasone, Budesonide, Mometasone Furoate, Fluocinolone, Flunisolide, Fluorometholone, Fluticasone, Dagrocorat, Dagrocorat Hydrochloride, Mapracorat, Fosdagrocorat, GSK9027, GSK866, AZD2906, GW-870086, BAY 1003803, ZK 216348, LEO 134310, and RU28362. In one embodiment, the glucocorticoid is Hydrocortisone 21-hemisuccinate. In one embodiment, the glucocorticoid is Hydrocortisone 21-hemisuccinate, which is present in the culture medium at a concentration with a range of 10 nM-100 μM. In one embodiment, the glucocorticoid in step (d) (Stage 3) is Hydrocortisone 21-hemisuccinate, which is present in the culture medium at a concentration of 10 μM. In one embodiment, the glucocorticoid in step (e) (Stage 4) is Hydrocortisone 21-hemisuccinate, which is

present in the culture medium at a concentration of 10 μM. In one embodiment, the glucocorticoid is Dexamethasone. In one embodiment, the glucocorticoid is Dexamethasone, which is present in the culture medium at a concentration with a range of 500 pM-5 M. In one embodiment, the glucocorticoid in step (d) (Stage 3) is Dexamethasone, which is present in the culture medium at a concentration of 100 nM. In one embodiment, the glucocorticoid in step (e) (Stage 4) is Dexamethasone, which is present in the culture medium at a concentration of 100 nM.

As used herein, the terms “cAMP response element-binding protein agonist” and “CREB agonist” refer to an agent that stimulates or increases signaling through or activity of CREB. In some examples, a CREB agonist is capable of (i) increasing phosphorylation of CREB or (ii) increasing expression of c-Fos. In some embodiments, the CREB agonist binds a CREB protein and stimulates or increases signaling through or activity of the CREB protein. In some embodiments, the CREB agonist is selected from the group consisting of Forskolin, cAMP, Dibutyryl cAMP, 8-Br-cAMP, cAMPS-Sp, and CW 008. In one embodiment, the CREB agonist is Forskolin. In one embodiment, the CREB agonist is Forskolin, which is present in the culture medium at a concentration within a range of 20 nM-2 mM. In one embodiment, the CREB agonist in step (d) (Stage 3) is Forskolin, which is present in the culture medium at a concentration of 20 μM. In one embodiment, the CREB agonist in step (e) (Stage 4) is Forskolin, which is present in the culture medium at a concentration of 20 μM.

As used herein, the term “antioxidant” refers to an agent that inhibits or decreases oxidation which produces free radicals. In some examples, an antioxidant is capable of (i) decreasing expression of reactive oxygen species (ROS), (ii) inhibiting ROS-induced expression of caspases, or (iii) decreasing expression of reactive nitrogen compounds. In some embodiments, the antioxidant directly reacts with free radicals. In some embodiments, the antioxidant inhibits the activity or expression of free radical generating enzymes. In some embodiments, the antioxidant stimulates the activity or expression of intracellular antioxidant enzymes. In some embodiments, the antioxidant is selected from the group consisting of N-Acetyl-L-Cysteine, Ascorbic Acid, Sodium Ascorbate, Glutathione, Ebselen, a-tocopherol, β-tocopherol, δ-tocopherol, γ-tocopherol, Lipoic Acid, Uric Acid, and Ubiquinol. In one embodiment, the antioxidant is N-Acetyl-L-Cysteine. In one embodiment, the antioxidant is N-Acetyl-L-Cysteine, which is present in the culture medium at a concentration within a range of 500 nM-2 M. In one embodiment, the antioxidant in step (d) (Stage 3) is N-Acetyl-L-Cysteine, which is present in the culture medium at a concentration of 1 mM. In one embodiment, the antioxidant is Ascorbic Acid. In one embodiment, the antioxidant is Ascorbic Acid, which is present in the culture medium at a concentration within a range of 250 pg/ml-25 mg/ml. In one embodiment, the antioxidant in step (d) (Stage 3) is Ascorbic Acid, which is present in the culture medium at a concentration of 60 μg/ml. In one embodiment, the antioxidant is Sodium Ascorbate. In one embodiment, the antioxidant is Sodium Ascorbate, which is present in the culture medium at a concentration within a range of 200 pg/ml-20 mg/ml. In one embodiment, the antioxidant in step (d) (Stage 3) is Sodium Ascorbate, which is present in the culture medium at a concentration of 50 μg/ml. In one embodiment, the antioxidant in step (e) (Stage 4) is Sodium Ascorbate, which is present in the culture medium at a concentration of 50 μg/ml.

As used herein, the term “Wnt antagonist” refers to an agent that inhibits or decreases signaling through or activity of a Frizzled family receptor (FZD). In some examples, a Wnt antagonist is capable of (i) reducing expression of Wnt, (ii) reducing expression of FZD, (iii) reducing phosphorylation of LRP5/6, (iv) reducing phosphorylation of DVL, or (v) increasing phosphorylation of β-catenin. In some embodiments, the Wnt antagonist prevents secretion of Wnt ligands by inhibiting Porcupine (PORCN). In some embodiments, the PORCN inhibitor is C59, IWP2, IWP4, WNT974, CGX1321, ETC-159, RXC004, or GNF-6231. In some embodiments, the Wnt antagonist is an agent targeting the ligands of FZD receptors. In some embodiments, the ligand-targeting agent is WIF-1 or Ipafricept. In some embodiments, the Wnt antagonist inhibits LRP5/6. In some embodiments, the LRP5/6 inhibitor is DKK1, BMD4503-2, or Salinomycin. In some embodiments, the Wnt antagonist inhibits DVL. In some embodiments, the DVL inhibitor is NSC 668036, FJ9, or 3289-8625. In some embodiments, the Wnt antagonist inhibits tankyrases. In some embodiments, the tankyrase inhibitor is IWR-1-endo, XAV939, WIKI4, JW55, JW74, NVP-TNKS656, LZZ-02, or TC-E5001. In some embodiments, the Wnt antagonist destabilizes the β-catenin/TCF complex. In some embodiments, the β-catenin/TCF complex-targeting agent is LF3, CCT036477, CCT251545, MSAB, KY1220, KY02111, FH535, Triptonide, KYA1797K, iCRT3, iCRT5, iCRT14, PNU-74654, PKF118-310, or Cardionogen 1. In some embodiments, the Wnt antagonist inhibits CREB binding protein (CBP). In some embodiments, the CBP inhibitor is ICG001, JW67, NLS-StAx-h, PRI-724, or GNE-781. In some embodiments, the Wnt antagonist inhibits MET receptor signaling. In some embodiments, the MET inhibitor is Capmatinib. In some embodiments, the Wnt antagonist inhibits TNIK. In some embodiments, the TNIK inhibitor is NCB-0846. In some embodiments, the Wnt antagonist inhibits casein kinase I (CK1). In some embodiments, the CK1 inhibitor is TAK715. In some embodiments, the Wnt antagonist inhibits pyruvate flavodoxin/ferredoxin oxidoreductases (PFORs). In some embodiments, the PFOR inhibitor is Nitazoxanide. In some embodiments, the Wnt antagonist binds a FZD and inhibits or decreases signaling through or activity of the FZD. In some embodiments, the Wnt antagonist is an antibody targeting FZDs. In some embodiments, the FZD-targeting antibody is Vantictumab. In some embodiments, the Wnt antagonist is an antagonist of FZDs. In some embodiments, the FZD antagonist is OTSA-101 or Fz7-21.

In some embodiments, the Wnt antagonist is selected from the group consisting of IWR-1-endo, C59, XAV939, WIKI4, JW55, JW74, NVP-TNKS656, LZZ-02, TC-E 5001, IWP2, IWP4, WNT974, CGX1321, ETC-159, RXC004, GNF-6231, WIF-1, Ipafricept, DKK1, BMD4503-2, Salinomycin, NSC 668036, FJ9, 3289-8625, LF3, CCT036477, CCT251545, MSAB, KY1220, KY02111, FH535, Triptonide, KYA1797K, iCRT3, iCRT5, iCRT14, PNU-74654, PKF118-310, Cardionogen 1, ICG-001, JW67, NLS-StAx-h, PRI-724, GNE-781, Capmatinib, NCB-0846, TAK715, Nitazoxanide, Vantictumab, OTSA-101, and Fz7-21. In one embodiment, the Wnt antagonist is IWR-1-endo. In one embodiment, the Wnt antagonist is IWR-1-endo, which is present in the culture medium at a concentration within a range of 500 pM-10 μM. In one embodiment, the Wnt antagonist in step (d) (Stage 3) is IWR-1-endo, which is present in the culture medium at a concentration of 500 nM. In one embodiment, the Wnt antagonist in step (e) (Stage 4) is IWR-1-endo, which is present in the culture medium at a concentration of 500 nM. In one embodiment, the Wnt antagonist is C59. In one embodiment, the Wnt antagonist is C59, which is present in the culture medium at a concentration within a range of 5 nM-100 μM. In one embodiment, the Wnt antagonist in step (e) (Stage 4) is C59, which is present in the culture medium at a concentration of 1 μM.

As used herein, the terms “transforming growth factor beta antagonist” and “TGF-β antagonist” refer to an agent that inhibits or decreases signaling through or activity of a TGF-β receptor family member. The TGF-β receptor may be a type I TGF-β receptor, which is an activin-like receptor (ALK) (e.g., ALK1, ALK2, ALK3, ALK4, ALK5, ALK6, or ALK7), a type II TGF-β receptor (e.g., TGFBR2, BMPR2, ACVR2A, ACVR2B, or AMHR2), or a type III TGF-β receptor (e.g., TGFBR3). In some examples, a TGF-β antagonist is capable of (i) reducing expression of TGF-β, (ii) reducing phosphorylation of the type I TGF-β receptor, or (iii) reducing phosphorylation of SMAD 2/3. In some embodiments, a TGF-β antagonist prevents TGF-β synthesis. In some embodiments, the TGF-β synthesis inhibitor is AP12009, AP11014, AP15012, or ISTH0036. In some embodiments, a TGF-β antagonist prevents TGF-β binding to the type II receptor. In some embodiments, the ligand-binding inhibitor is Fresolimumab, Lerdelimumab, GC1008, 2G7, 1D11, CAT-192, or LY2382770. In some embodiments, the TFG-β antagonist binds a type I or type II receptor and inhibits or decreases signaling through or activity of the type I or type II receptor. In some embodiments, a TGF-β antagonist is an antibody targeting the type II receptor. In some embodiments, the type II receptor-targeting antibody is LY3022859. In some embodiments, a TGF-β antagonist is an inhibitor of the type I receptor kinase activity. In some embodiments, the type I receptor kinase inhibitor is SB431542, A 83-01, GW788388, SB505124, SB525334, TP0427736, RepSox, SD-208, Galunisertib, IN-1130, Ki 26894, LY2109761, LY2157299, LY550410, PF-03446962, or TEW-7197.

In some embodiments, the TGF-β antagonist is selected from the group consisting of SB431542, A 83-01, GW788388, SB505124, SB525334, TP0427736, RepSox, SD-208, Galunisertib, IN-1130, Ki 26894, LY2109761, LY2157299, LY550410, PF-03446962, TEW-7197, AP12009, AP11014, AP15012, ISTH0036, Fresolimumab, Lerdelimumab, GC1008, 2G7, 1D11, CAT-192, LY2382770, and LY3022859. In one embodiment, the TGF-β antagonist is SB431542. In one embodiment, the TGF-β antagonist is present in the culture medium at a concentration within a range of 10 nM-200 μM. In one embodiment, the TGF-β antagonist in step (d) (Stage 3) is SB431542, which is present in the culture medium at a concentration of 10 μM. In one embodiment, the TGF-β antagonist in step (e) (Stage 4) is SB431542, which is present in the culture medium at a concentration of 10 μM.

As used herein, the terms “hepatocyte growth factor agonist” and “HGF agonist” refer to an agent that stimulates or increases signaling through or activity of an HGF receptor (e.g., c-MET receptor). In some examples, an HGF agonist is capable of (i) increasing phosphorylation of c-MET, (ii) increasing phosphorylation of GAB1, (iii) increasing phosphorylation of AKT, or (iv) increasing phosphorylation of ERK. In some embodiments, the HGF agonist binds an HGF receptor and stimulates or increases signaling through or activity of the HGF receptor. In some embodiments, the HGF agonist is selected from the group consisting of HGF, Dihexa, NK1, NK2, Fosgonimeton, and Terevalefim. In one embodiment, the HGF agonist is HGF. In one embodiment, the HGF agonist is HGF, which is present in the culture medium at a concentration within a range of 100 pg/ml-10 μg/ml. In one embodiment, the HGF agonist in step (d) (Stage 3) is HGF, which is present in the culture medium at a concentration of 10 ng/ml. In one embodiment, the HGF agonist is Dihexa. In one embodiment, the HGF agonist is Dihexa, which is present in the culture medium at a concentration within a range of 500 pg/ml-1 μg/ml. In one embodiment, the HGF agonist in step (d) (Stage 3) is Dihexa, which is present in the culture medium at a concentration of 100 nM. In one embodiment, the HGF agonist in step (e) (Stage 4) is Dihexa, which is present in the culture medium at a concentration of 100 nM.

As used herein, the term “Notch antagonist” refers to an agent that inhibits or decreases signaling through or activity of a Notch receptor (e.g., NOTCH1, NOTCH2, NOTCH3, and NOTCH4). In some examples, a Notch antagonist is capable of (i) reducing cell surface expression of Notch receptors, (ii) reducing expression of Notch Intracellular Domain (NICD) induced by Delta-like 4(DLL 4 ), (iii) reducing expression of NOTCH1, or (iv) reducing the expression of HES-1 induced by DLL4. In some embodiments, the Notch antagonist is an inhibitor of membrane trafficking of Notch receptors. In some embodiments, the inhibitor of membrane trafficking of Notch receptors is FLI-06, Thapsigargin, or CAD204520. In some embodiments, the Notch antagonist is an agent targeting the ligands of Notch receptors. In some embodiments, the agent targeting the ligands of Notch receptors is Tangeretin, Bruceine D, 15D11, Enoticumab, Demcizumab, ABT-165, or Navicixizumab. In some embodiments, the Notch antagonist is an inhibitor of A Disintegrin and Metalloproteases (ADAMs). In some embodiments, the inhibitor of ADAMs is Marimastat or ZLDI-8. In some embodiments, the Notch antagonist is a y-secretase inhibitor (GSI). In some embodiments, the GSI is DAPT, GSI-XX, BMS 299897, BMS 433796, BMS 906024, BMS 986115, Compound E, Compound W, Compound 18, DBZ, DFK-167, L-685458, LY 3039478, LY411575, LY 450139, LY900009, MK-0752, MRK 003, MRK 560, PF 3084014, PF 3084014 Hydrobromide, Z-IL-CHO, Avagacestat, Begacestat, JLK6, AL101, or RO4929097. In some embodiments, the Notch antagonist is a transcription blocker. In some embodiments, the transcription blocker is IMR-1, IMR-1A, CB-103, or RIN1. In some embodiments, the Notch antagonist binds a Notch receptor and inhibits or decreases signaling through or activity of the Notch receptor. In some embodiments, the Notch antagonist is an antibody targeting one or more types of Notch receptors. In some embodiments, the antibody targeting one or more types of Notch receptors is Brontictuzumab, Tarextumab, or PF-06650808.

In some embodiments, the Notch antagonist is selected from the group consisting of DAPT, GSI-XX, BMS 299897, BMS 433796, BMS 906024, BMS 986115, Compound E, Compound W, Compound 18, DBZ, DFK-167, L-685458, LY 3039478, LY 411575, LY 450139, LY 900009, MK-0752, MRK 003, MRK 560, PF 3084014, PF 3084014 Hydrobromide, Z-IL-CHO, Avagacestat, Begacestat, JLK6, AL101, RO 4929097, FLI-06, Thapsigargin, CAD204520, Tangeretin, Bruceine D, 15D11, Enoticumab, Demcizumab, ABT-165, Navicixizumab, Marimastat, ZLDI-8, IMR-1, IMR-1A, CB-103, RIN1, Brontictuzumab, Tarextumab, and PF 06650808. In one embodiment, the Notch antagonist is DAPT. In one embodiment, the Notch antagonist is DAPT, which is present in the culture medium at a concentration within a range of 10 nM-100 μM. In one embodiment, the Notch antagonist in step (e) (Stage 4) is DAPT, which is present in the culture medium at a concentration of 1 μM.

As used herein, the terms “bone morphogenetic protein antagonist” and “BMP antagonist” refer to an agent that inhibits or decreases signaling through or activity of a BMP receptor. The BMP receptor may be a type I BMP receptor (e.g., ALK1, ALK2, ALK3, or ALK6), or a type II BMP receptor (e.g., BMPR2, activin A receptor type II (ActR2), or activin A receptor type IIB (ActR2B)). In some examples, a BMP antagonist is capable of (i) reducing phosphorylation of Smad1/5/8 induced by BMP, (ii) reducing expression of Smad1/5/8, (iii) reducing phosphorylation of ERK induced by BMP, or (iv) reducing expression of Id1. In some embodiments, the BMP antagonist binds BMPs. In some embodiments, the BMP-binding agent is Follistatin, Follistatin-like 1, Noggin, Chordin, Ventroptin, Twisted gastrulation, Dan, Cerberus, PRDC, Dante, Caronte, Gremlin, or Sclerostin. In some embodiments, the BMP antagonist binds a BMP receptor and inhibits or decreases signaling through or activity of the BMP receptor. In some embodiments, the BMP antagonist is a selective BMP type I receptor inhibitor. In some embodiments, the BMP type I receptor inhibitor is LDN193189, Dorsomorphin, DMH-1, DMH-2, ML 347, LDN212854, LDN214117, or K02288.

In some embodiments, the BMP antagonist is selected from the group consisting of LDN193189, Dorsomorphin, DMH-1, DMH-2, ML 347, LDN212854, LDN214117, K02288, Follistatin, Follistatin-like 1, Noggin, Chordin, Ventroptin, Twisted Gastrulation, Dan, Cerberus, PRDC, Dante, Caronte, Gremlin, and Sclerostin. In one embodiment, the BMP antagonist is LDN193189. In one embodiment, the BMP antagonist is LDN193189, which is present in the culture medium at a concentration within a range of 500 pM-2.5 μM. In one embodiment, the BMP antagonist in step (e) (Stage 4) is LDN193189, which is present in the culture medium at a concentration of 100 nM.

As used herein, the term “hepatocyte function enhancer” refers to an agent that enhances hepatocyte functions and promotes differentiation of iPSC-derived hepatocytes towards a more mature phenotype. In some embodiments, the hepatocyte function enhancer is FH1 or FPH1. In one embodiment, the hepatocyte function enhancer is FH1. In one embodiment, the hepatocyte function enhancer is FH1, which is present in the culture medium at a concentration within a range of 10 nM-2.5 mM. In one embodiment, the hepatocyte function enhancer in step (e) (Stage 4) is FH1, which is present in the culture medium at a concentration of 15 μM.

When an agonist or antagonist is used in more than one step of the method, in one embodiment it is the same agonist or antagonist that is used for each step in which the agent is present in the culture media. In another embodiment, different agonists or antagonists that affect the same signaling pathway are used in different steps of the method. For example, for the HDAC inhibitor used in steps (c) and (e) (Stages 2 and 4), in one embodiment the same HDAC inhibitor is used in steps (c) and (e). In another embodiment, different HDAC inhibitors are used in steps (c) and (e). Similarly, for the glucocorticoid used in steps (d) and (e) (Stages 3 and 4), in one embodiment the same glucocorticoid is used in steps (d) and (e). In another embodiment, different glucocorticoids are used in steps (d) and (e). Similarly, for the CREB agonist used in steps (d) and (e) (Stages 3 and 4), in one embodiment the same CREB agonist is used in steps (d) and (e). In another embodiment, different CREB agonists are used in steps (d) and (e).

Similarly, for the antioxidant used in steps (d) and (e) (Stages 3 and 4), in one embodiment the same antioxidant is used in steps (d) and (e). In another embodiment, different antioxidants are used in steps (d) and (e). Similarly, for the Wnt antagonist used in steps (d) and (e) (Stages 3 and 4), in one embodiment the same Wnt antagonist is used in steps (d) and (e). In another embodiment, different Wnt antagonists are used in steps (d) and (e). Similarly, for the TGF-β antagonist used in steps (d) and (e) (Stages 3 and 4), in one embodiment the same TGF-β antagonist is used in steps (d) and (e). In another embodiment, different TGF-β antagonists are used in steps (d) and (e). Similarly, for the HGF agonist used in steps (d) and (e) (Stages 3 and 4), in one embodiment the same HGF agonist is used in steps (d) and (e). In another embodiment, different HGF agonists are used in steps (d) and (e).

When an agonist or antagonist is used in more than one step of the method, in one embodiment it is the same concentration of the same agonist or antagonist that is used for each step in which the agent is present in the culture media. In another embodiment, different concentrations of the same agonist or antagonist are used in different steps of the method. For example, for the HDAC inhibitor used in steps (c) and (e) (Stages 2 and 4), in one embodiment the same concentration of the same HDAC inhibitor is used in steps (c) and (e). In another embodiment, different concentrations of the same HDAC inhibitor are used in steps (c) and (e). Similarly, for the glucocorticoid used in steps (d) and (e) (Stages 3 and 4), in one embodiment the same concentration of the same glucocorticoid is used in steps (d) and (e). In another embodiment, different concentrations of the same glucocorticoid are used in steps (d) and (e).

Similarly, for the CREB agonist used in steps (d) and (e) (Stages 3 and 4), in one embodiment the same concentration of the same CREB agonist is used in steps (d) and (e). In another embodiment, different concentrations of the same CREB agonist are used in steps (d) and (e). Similarly, for the antioxidant used in steps (d) and (e) (Stages 3 and 4), in one embodiment the same concentration of the same antioxidant is used in steps (d) and (e). In another embodiment, different concentrations of the same antioxidant are used in steps (d) and (e). Similarly, for the Wnt antagonist used in steps (d) and (e) (Stages 3 and 4), in one embodiment the same concentration of the same Wnt antagonist is used in steps (d) and (e). In another embodiment, different concentrations of the same Wnt antagonist are used in steps (d) and (e). Similarly, for the TGF-β antagonist used in steps (d) and (e) (Stages 3 and 4), in one embodiment the same concentration of the same TGF-β antagonist is used in steps (d) and (e). In another embodiment, different concentrations of the same TGF-β antagonist are used in steps (d) and (e). Similarly, for the HGF agonist used in steps (d) and (e) (Stages 3 and 4), in one embodiment the same concentration of the same HGF agonist is used in steps (d) and (e). In another embodiment, different concentrations of the same HGF agonist are used in steps (d) and (e).

### III. Culture Conditions

In combination with the chemically-defined and optimized culture media described in subsection II above, the methods of generating endodermal cells, midgut cells, hepatoblasts, and hepatocytes of the disclosure utilize standard culture conditions established in the art for cell culture. For example, cells can be cultured at 37° C. and under 5% O₂ and 5% CO₂ conditions. Cells can be cultured in standard culture vessels or plates, such as 96-well plates. In certain embodiments, the starting iPSCs are adhered to plates, preferably coated with an extracellular matrix material such as vitronectin. In one embodiment, the stem cells are cultured on a vitronectin-coated culture surface (e.g., vitronectin coated 96-well plates).

iPSCs can be cultured in commercially available media prior to differentiation. For example, stem cells can be cultured for at least one day in Essential 8 Flex media (Thermo Fisher #A 2858501) prior to the start of the differentiation protocol. In a non-limiting exemplary embodiment, stem cells are passaged onto vitronectin (Thermo Fisher #A 14700)-coated 96-well plates at 150,000 cells/cm² density and cultured for one day in Essential 8 Flex media prior to differentiation.

To begin the differentiation protocol, the media the stem cells are being cultured in is changed to a basal differentiation media that has been supplemented with signaling pathway agonists or antagonists as described above in subsection II. A basal differentiation media can include, for example, a commercially-available base media supplemented with additional standard culture media components needed to maintain cell viability and growth, but lacking serum (the basal differentiation media is a serum-free media) or any other exogenously-added growth factors, such as FGF2 or HGF. In a non-limiting exemplary embodiment, a basal differentiation media contains 1× IMDM (Thermo Fisher #12440046), 1×F12 (Thermo Fisher #11765047), poly(vinyl alcohol) (Sigma #p8136) at 1 mg/ml, chemically-defined lipid concentrate (Thermo Fisher #11905031) at 1%, 1-thioglycerol (Sigma #M6145) at 450 μM, insulin (Sigma #11376497001) at 0.7 ug/ml, and transferrin (Sigma #10652202001) at 15 ug/ml. The culture media typically is changed regularly to fresh media. For example, in one embodiment, media is changed every 24 hours.

To generate endodermal cells, midgut cells, hepatoblasts, and hepatocytes, the starting stem cells are cultured in the optimized culture media for sufficient time for cellular differentiation and expression of committed endodermal-, midgut-, hepatoblast-, and hepatocyte-associated markers. It has been discovered that culture of iPSCs in a four-stage method, one optimized for the generation of endodermal cells, a second optimized for the generation of midgut cells, a third optimized for the generation of hepatoblasts, and a fourth optimized for the generation of hepatocytes, can lead to the production of hepatocytes in as little as 24 days of culture. The culture period for the first stage (leading to endodermal cells) is days 0-2, the culture period for the second stage (leading to midgut cells) is days 2-7, the culture period for the third stage (leading to hepatoblasts) is days 7-17, and the culture period for the fourth stage (leading to hepatocytes) is days 17-24.

Accordingly, in the first stage of the method, which generates endodermal cells, also referred to herein as “steps (a) and (b)” or “Stage 1”, iPSCs are cultured in the Stage 1-optimized culture media on days 0-2, or starting on day 0 and continuing through day 2, or starting on day 0 and continuing for 48 hours (two days), or starting on day 0 and continuing for at least 24 hours, at least 48 hours, at least 72hours, at least 96 hours, at least 120 hours, at least 144 hours, at least 168 hours, at least 192 hours, at least 216 hours, or at least 240 hours, or starting on day 0 and continuing for 24 hours, 48 hours, 72 hours, 96 hours, 120 hours, 144 hours, 168 hours, 192 hours, 216 hours, or 240 hours.

Accordingly, in the second stage of the method, which generates midgut cells, also referred to herein as “step (c)” or “Stage 2”, the endodermal cells generated in steps (a) and (b) are further cultured in the Stage 2-optimized culture media on days 2-7, or starting on day 2 and continuing through day 7, or starting on day 2 and continuing for 120 hours (five days), or starting on day 2 and continuing for at least 24 hours, at least 48 hours, at least 72 hours, at least 96 hours, at least 120 hours, at least 144 hours, at least 168 hours, at least 192 hours, at least 216 hours, or at least 240 hours, or starting on day 2 and continuing for 24 hours, 48 hours, 72 hours, 96 hours, 120 hours, 144 hours, 168 hours, 192 hours, 216 hours, or 240 hours.

Accordingly, in the third stage of the method, which generates hepatoblasts, also referred to herein as “step (d)” or “Stage 3”, the midgut cells generated in step (c) are further cultured in the Stage 3-optimized culture media on days 7-17, or starting on day 7 and continuing through day 17, or starting on day 7 and continuing for 240 hours (10 days), or starting on day 7 and continuing for at least 48 hours, at least 72 hours, at least 96 hours, at least 120 hours, at least 144 hours, at least 168 hours, at least 192 hours, at least 216 hours, or at least 240 hours, or starting on day 7 and continuing for 48 hours, 72 hours, 96 hours, 120 hours, 144 hours, 168 hours, 192 hours, 216 hours, or 240 hours.

Accordingly, in the fourth stage of the method, which generates hepatocytes, also referred to herein as “step (e)” or “Stage 4”, the hepatoblasts generated in step (d) are further cultured in the Stage 4-optimized culture media on days 17-24, or starting on day 17 and continuing through day 24, or starting on day 17 and continuing for 168 hours (seven days), or starting on day 17 and continuing for at least 48 hours, at least 72 hours, at least at least 96 hours, at least 120 hours, at least 144 hours, or at least 168 hours, or starting on day 17 and continuing for 48 hours, 72 hours, 96 hours, 120 hours, 144 hours, or 168 hours.

### IV. Uses

The methods and compositions of the disclosure for generating endodermal cells, midgut cells, hepatoblasts, and hepatocytes allow for efficient and robust availability of these cell populations for a variety of uses.

The methods and compositions of the disclosure can be used in the study of physiological roles of hepatocytes in healthy individuals. Hepatocytes play essential roles in the production and secretion of several circulating molecules (e.g., albumin and A1AT), detoxification of compounds or drugs, removal of compounds from circulation, nitrogenous waste removal through urea generation, regulation of metabolism across the organism, glucose homeostasis, glycogen storage, production of coagulation factors, and production of bile salt precursors.

The methods and compositions of the disclosure can also be used in the study of toxicity. While animal-derived hepatocytes and in vivo preclinical studies do not represent human biology and often fail to predict the clinical outcomes, iPSC-derived hepatocytes are ideal for in vitro toxicity studies as they represent a renewable and consistent source for human hepatocytes. Specifically, iPSC-derived hepatocytes are ideal for Absorption, Distribution, Metabolism, Excretion (ADME), Drug Metabolism and Pharmacokinetics (DMPK), and toxicology studies.

Moreover, the methods and compositions of the disclosure can be used for research on tissue crosstalk, which occurs through the circulatory system. Several tissues, such as the intestines, pancreas, adipose tissue, and kidney, communicate with the liver in this manner. Accordingly, iPSC-derived hepatocytes can be used in the organ-on-a-chip models for the study of tissue crosstalk.

Further, the methods and compositions of the disclosure are useful for the study of liver diseases, such as steatosis, NAFLD, ALD, NASH, metabolic disorder, mitochondrial dysfunction, insulin resistance, and cirrhosis.

Lastly, the methods and compositions of the disclosure can be used for engineering liver tissue.

### V. Compositions

In other aspects, the disclosure provides compositions related to the methods of generating endodermal cells, midgut cells, hepatoblasts, and hepatocytes, including culture media and isolated cell cultures, as well as populations of endodermal cells, midgut cells, hepatoblasts, and hepatocytes.

In one aspect, the disclosure provides a culture medium for obtaining endodermal cells, the media comprising a GSK3B inhibitor. Suitable agents, and concentrations therefor, include those described in subsection II.

In one aspect, the disclosure provides a culture medium for obtaining midgut cells, the media comprising an HDAC inhibitor, a RA agonist, and an FGFR (e.g., FGFR1) agonist. Suitable agents, and concentrations therefor, include those described in subsection II.

In one aspect, the disclosure provides a culture medium for obtaining hepatoblasts, the media comprising a glucocorticoid, a CREB agonist, an antioxidant, a Wnt antagonist, a TGF-β antagonist, and an HGF agonist. Suitable agents, and concentrations therefor, include those described in subsection II.

In one aspect, the disclosure provides a culture medium for obtaining hepatocytes, the media comprising an HDAC inhibitor, a glucocorticoid, a CREB agonist, an antioxidant, a Wnt antagonist, a TGF-β antagonist, an HGF agonist, a Notch antagonist, a BMP antagonist, and a hepatocyte function enhancer. Suitable agents, and concentrations therefor, include those described in subsection II.

In one aspect, the disclosure provides an isolated cell culture of endodermal cells, the culture comprising endodermal cells in a culture medium comprising a GSK3β inhibitor. Suitable agents, and concentrations therefor, include those described in subsection II.

In one aspect, the disclosure provides an isolated cell culture of midgut cells, the culture comprising midgut cells cultured in a culture medium comprising an HDAC inhibitor, a RA agonist, and an FGFR (e.g., FGFR1) agonist. Suitable agents, and concentrations therefor, include those described in subsection II.

In one aspect, the disclosure provides an isolated cell culture of hepatoblasts, the culture comprising hepatoblasts cultured in a culture medium comprising a glucocorticoid, a CREB agonist, an antioxidant, a Wnt antagonist, a TGF-β antagonist, and an HGF agonist. Suitable agents, and concentrations therefor, include those described in subsection II.

In one aspect, the disclosure provides an isolated cell culture of 50 million to 50 billion hepatocytes, the culture comprising hepatocytes cultured in a culture medium comprising an HDAC inhibitor, a glucocorticoid, a CREB agonist, an antioxidant, a Wnt antagonist, a TGF-β antagonist, an HGF agonist, a Notch antagonist, a BMP antagonist, and a hepatocyte function enhancer. Suitable agents, and concentrations therefor, include those described in subsection II.

In one aspect, the disclosure provides endodermal cells generated by a method of the disclosure (i.e., steps (a) and (b) or Stage 1 of the culture protocol).

In one aspect, the disclosure provides midgut cells generated by a method of the disclosure (i.e., steps (a)-(c) or Stages 1 and 2 of the culture protocol).

In one aspect, the disclosure provides hepatoblasts generated by a method of the disclosure (i.e., steps (a)-(d) or Stages 1-3 of the culture protocol).

In one aspect, the disclosure provides hepatocytes generated by a method of the disclosure (i.e., steps (a)-(e) or Stages 1-4 of the culture protocol).

In one aspect, the disclosure provides an isolated cell culture comprising 50 million to 50 billion engineered hepatocytes.

### EXAMPLES

### Example 1. Protocol Development for the Generation of Hepatocytes From iPSCs

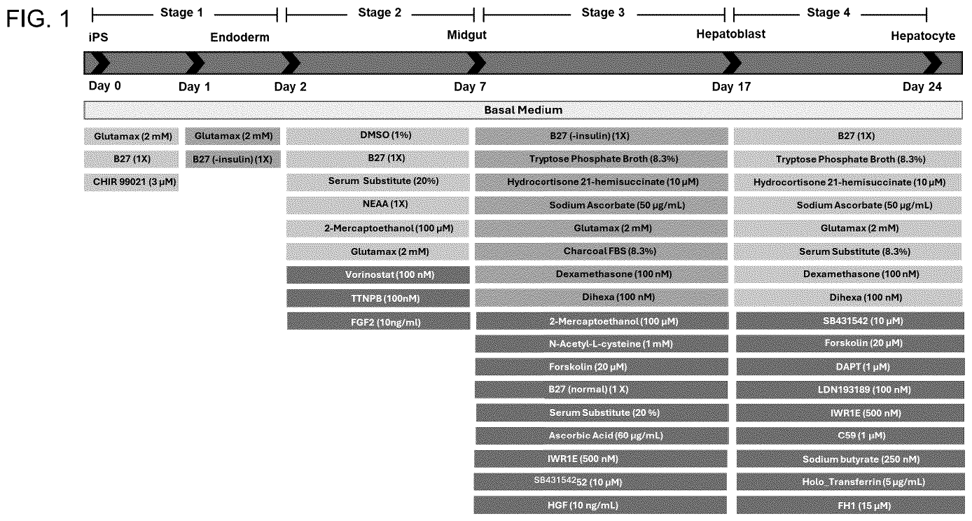

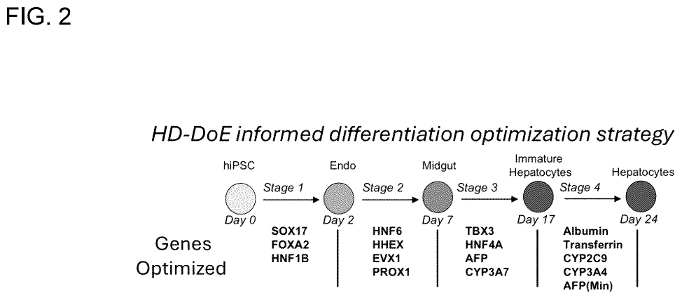

The invention being described here is outlined in FIG. 1. The components and concentrations of reagents used at the discrete different stages of the process are all under the appropriate protocol stage as outlined at the top of the schematic. Reagents defined through HD-DoE modelling experiments are Vorinostat, TTNPB, and FGF2 in stage 2; 2-Mercaptoethanol, N-Acetyl-L-cysteine Forskolin, B27, Serum Substitute, Ascorbic Acid, IWR1E, SB431542, and HGF in stage 3; SB431542, Forskolin, DAPT, LDN193189, IWR1E, C59, Sodium butyrate, Holo_Transferrin, and FH1 in stage 4. The differentiation approach taken is outlined in FIG. 2 with the genes that were optimized for at each stage listed beneath the individual stages. An HD-DoE-informed differentiation optimization strategy was employed for the development of this protocol. All target genes shown in the schematic were optimized for maximal expression, with the exception of AFP in stage 4, which was minimized in stage 4.

### Optimizing for Robust Endoderm Induction From an iPSC

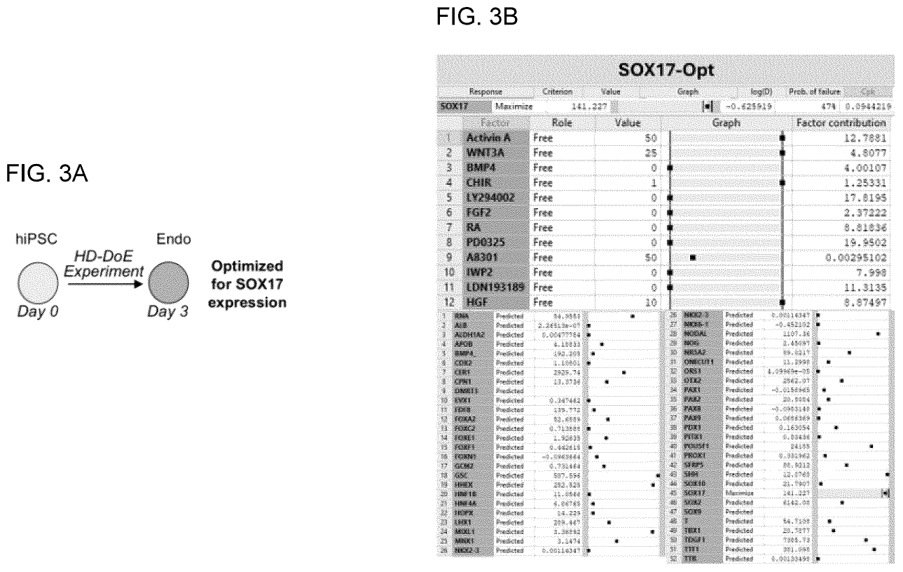

It is commonly accepted that all endodermal lineages are derived from a single progenitor cell type, commonly referred to as endoderm. The most commonly associated genes with this cellular state are the co-expression of FOXA2 and SOX17. For this reason, the initial efforts focused on using the HD-DoE-informed approach to maximize for the differentiation SOX17 expression on a forward differentiating iPSC culture (FIGS. 3A-3B). In this effort, a modeling experiment assaying the small molecule agonists of TGF-β, Wnt, BMP, FGFR, and retinoic acid (RA) pathways was set up. These are all commonly used pathways to initialize differentiation in iPSC cultures. In addition to the agonists of these pathways used, small molecule inhibitors of these same pathways were included in the perturbation matrix. The combination of pathway agonism and antagonism enables us to have full control over the different pathways within the in-silico models generated from the data. The results for the optimal induction of SOX17 are shown (FIGS. 3A-3B) and show that both the TGF-β and Wnt signaling pathways are involved in the activation of SOX17.

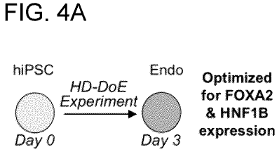

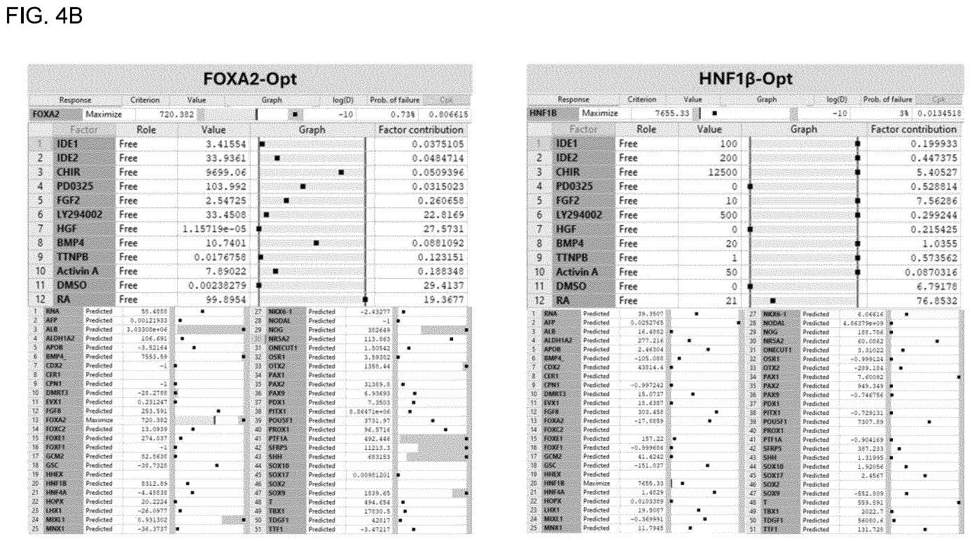

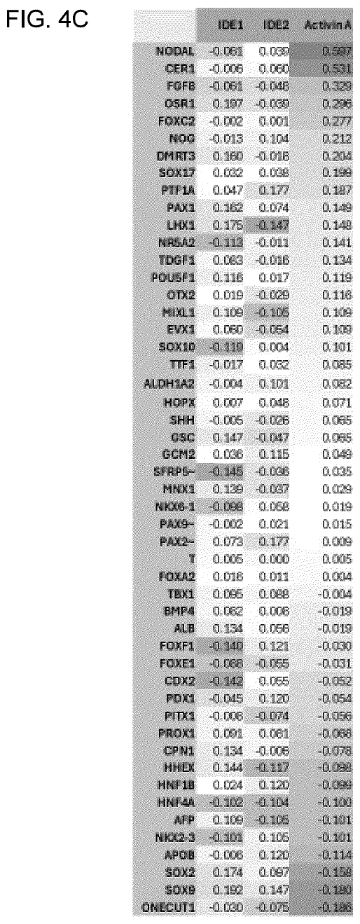

Since protein agonists are both costly and prone to degradation, resulting in decreased efficacy over time, a series of small molecules agonists was screened to potentially replace protein agonists of the TGF-β pathway using IDE1 and IDE2 (FIGS. 4A-4B). Including these components within an HD-DoE modeling experiment allowed optimal endoderm induction through the maximization of the endoderm targets FOXA2 and HNF1B. These genes had contrasting regulatory inputs with FOXA2 only responding to retinoid acid and Wnt pathway agonists, while HNF1B induction was shown to respond mostly to retinoid stimulation with a FC value of 76.9, but only with a predicted low concentration of 21 nM. Interestingly the TGF-β pathway, the most commonly used for endoderm induction, was shown to have little positive effect in activating these genes and FOXA2 was predicted to have a negative effect on its induction. Both IDE1 and IDE2 showed a poor correlation to the effects of activin A (FIG. 4C).

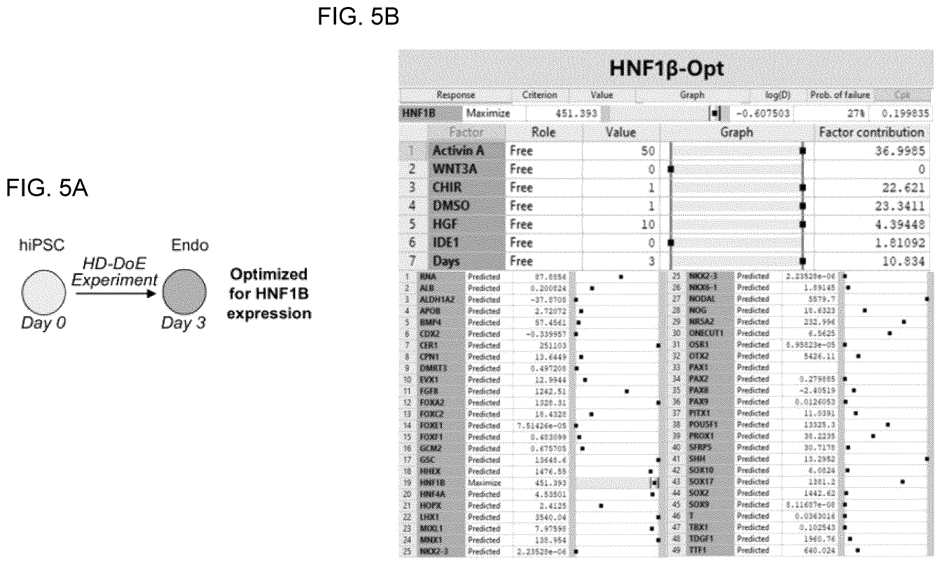

Prior art suggests two opposing methods for inducing endoderm from a stem cell precursor state. To evaluate which endoderm induction method works best, effectors from both methods were combined into a single modeling experiment. Since the opposing methods differed in the length of the induction period, the timing of the induction was included in the modeling experiment (FIG. 5A), enabling the evaluation of length of exposure within the opposing induction methods. The optimal conditions for inducing HNF1B within this perturbation matrix are shown (FIG. 5B).

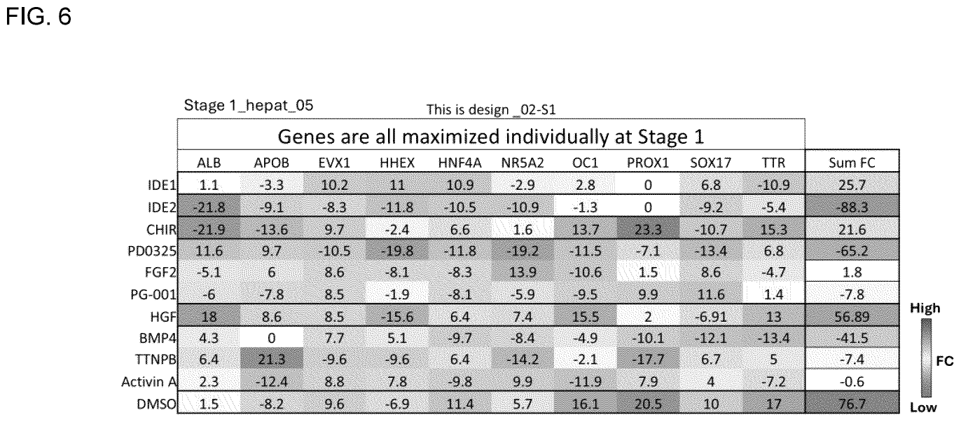

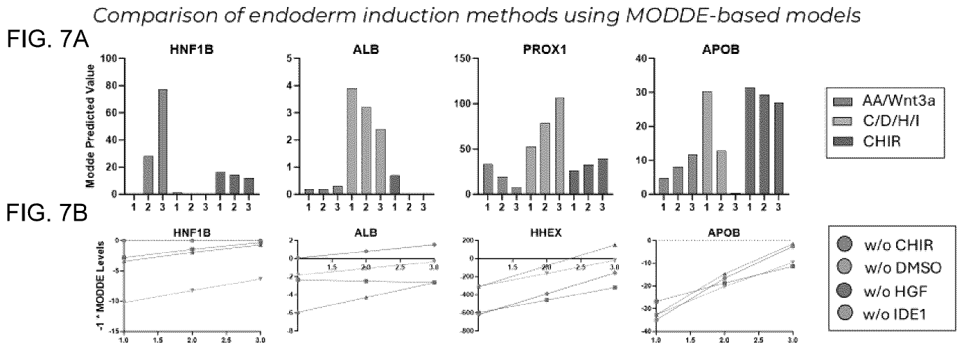

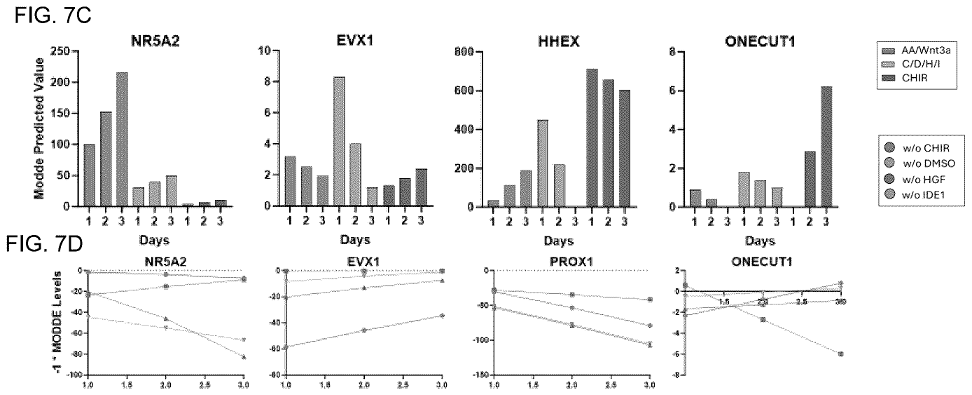

To take a broader approach to determine optimal conditions for endoderm induction, several endodermal genes were maximized and evaluated for co-regulatory inputs. A list of 11 factors was tested in this approach and are listed in the left column of the heatmap (FIG. 6). All the genes listed at the top were optimized for maximum expression using MODDE software and the differentiation inducing reagents'impact on the genes induction are listed as factor contribution (FC) numbers. Larger FC numbers are indicative of the overall strength of the effect and positive FC numbers represent positive effects while negative FC numbers indicate negative effects on the induction of the gene being investigated. The overall effect of the genes measured were summed across all genes investigated with the average FC presented on the right side of the heatmap. Through this method it was shown that there were four factors identified that had an overall positive effect on inducing hepatic competence. The pathways indicated for this induction are TGF-β pathway (IDE1), the Wnt pathway (CHIR), the c-MET pathway (HGF) and HDAC inhibition (DMSO). It should be noted that while the small molecule agonist IDE1 showed a positive effect, neither the IDE2 nor activin A had an overall stimulatory effect on inducing all the hepatic specific endodermal gene assayed in this manner, suggesting that IDE1 may have broader effects than just a TGF-β agonist. To evaluate the induction properties of the combinatorial effects of these four small molecules (CHIR/DMSO/HGF/IDE1 hence forward referred to as C/D/H/I), the C/D/H/I induction method was directly compared in silico to the well-established methods using a 3-day induction of AA/WNT3a and the commonly used 1-day induction using CHIR (FIGS. 7A-7D). Timing of the induction was included in the in silico models to account for the different lengths of exposure between the two literature-based approaches. Using the C/D/H/I induction method, it was shown that a 1-day of induction induced maximal expression of several hepatic genes, including ALB, APOB, EVX1, HHEX, and ONECUT1. To assess the overall effects of the individual component in the C/D/H/I approach, computer models showing the effects of removing each of the small molecules independently were generated and are provided beneath graphs evaluating the different approaches (FIGS. 7B and 7D). A positive slope in these models indicates a contribution for inducing the gene being investigated. Notably the removal of HGF and IDE1 negatively affected the induction of NR5A2 and ONECUT1, while the removal of any of the components negatively affected the induction of PROX1.

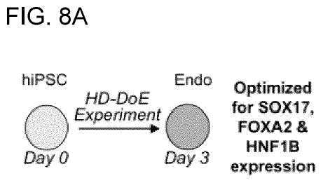

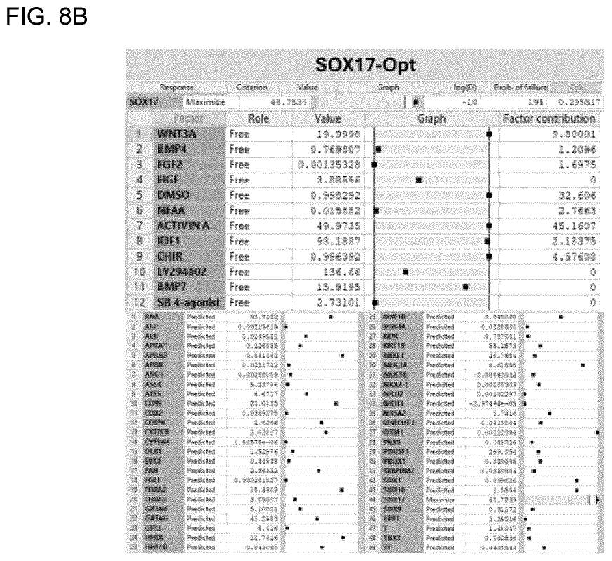

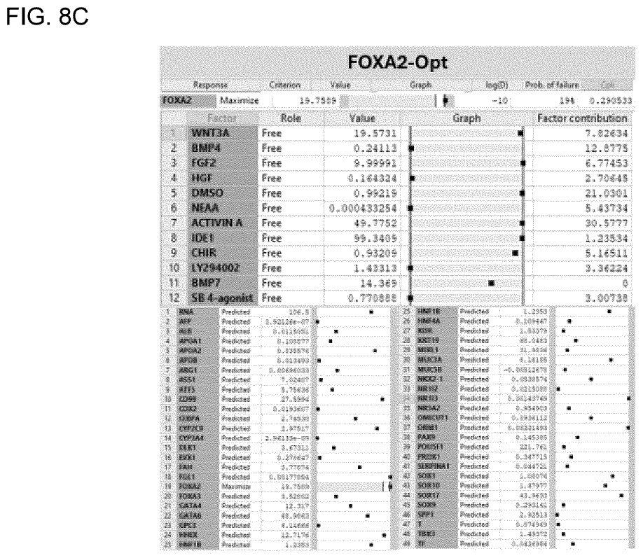

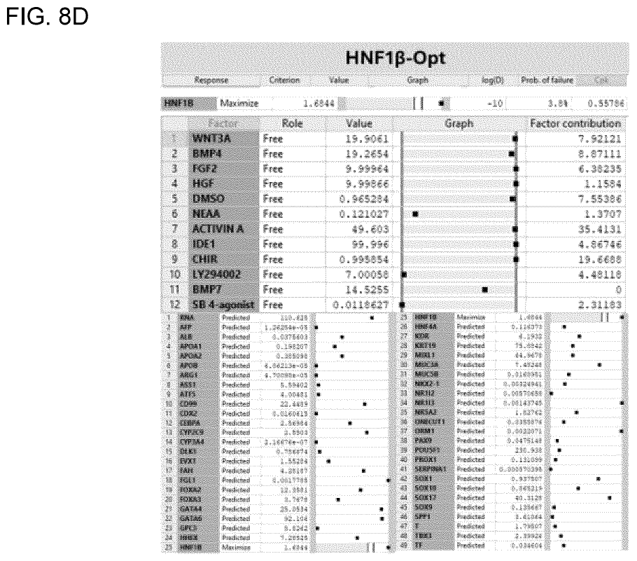

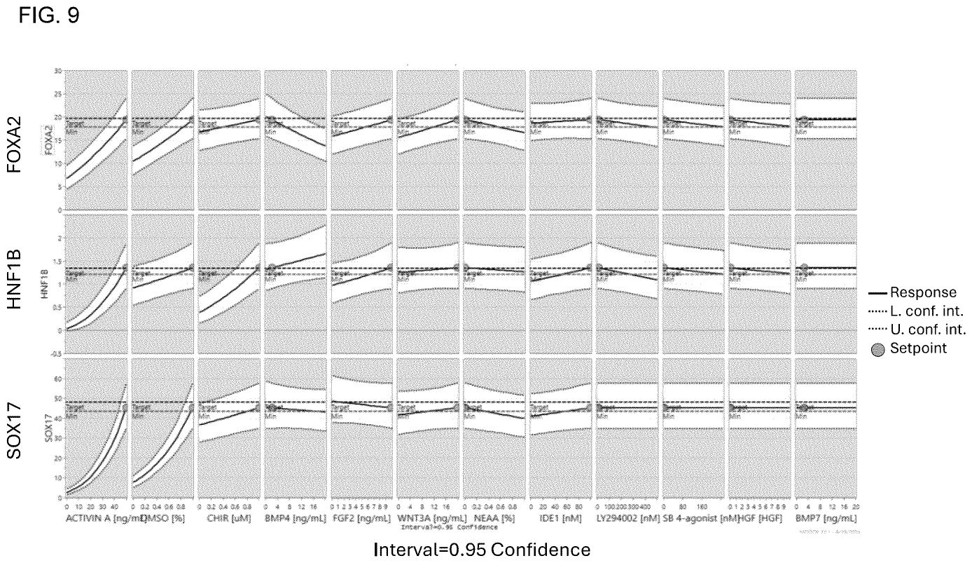

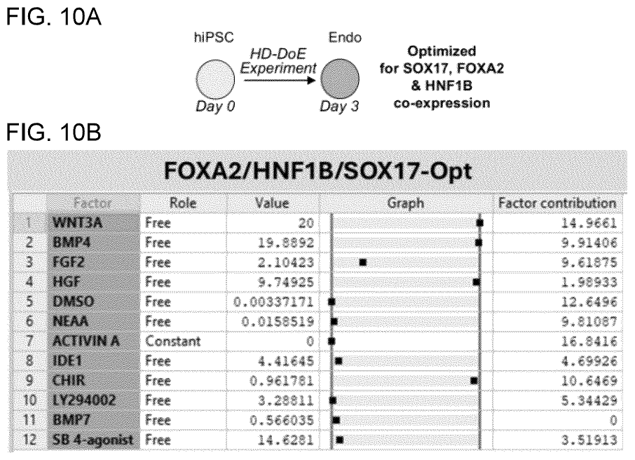

The final efforts of modeling endoderm induction through HD-DoE focused on the co-expression of three crucial endoderm genes that, when co-expressed, define an endoderm state (FIGS. 8A-8D). The maximization of SOX17, FOXA2, and HNF1B are shown to have co-regulatory inducers with WNT3a, DMSO, activin A, and CHIR being indicated as inducers of all three genes (FIGS. 8B-8D). The involvement of these pathways was further confirmed using dynamic profiling of the three genes (FIG. 9). However, little synergy between these compounds was shown to exist, suggesting that the effects of the compounds were independent of each other and that any of the compounds could be used independently or in combination. Throughout the series of stage 1 modeling experiments it was demonstrated that the greatest variability of the reagents used was observed with activin A, while CHIR consistently achieved the same inductive potential across experiments. Therefore, TGF-β signaling was eliminated from models to evaluate the cruciality of the Wnt pathway for endoderm induction (FIGS. 10A-10B). Eliminating the variability of activin A used in the in silico analysis resulted in a sequential model for the optimization of FOXA2/HNF1B/SOX17. This series of models showed that the two compounds with the greatest positive effect in inducing the co-expression of FOXA2/HNF1B/SOX17 were WNT3a and CHIR, both of which are Wnt pathway agonists (FIG. 10B). Additionally, it was noted that for this activation to occur it was significantly important that activin A was not included in the reaction. This suggests that this endodermal induction was occurring through a process that is distinctive from the TGF-β signaling induction.

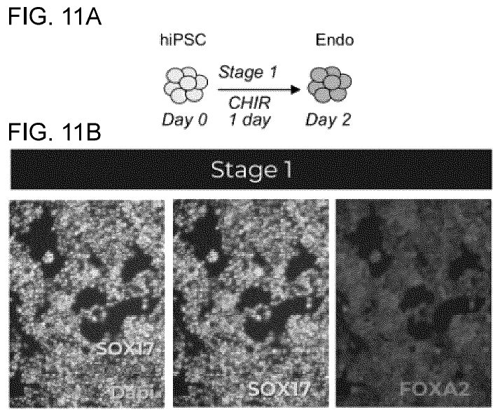

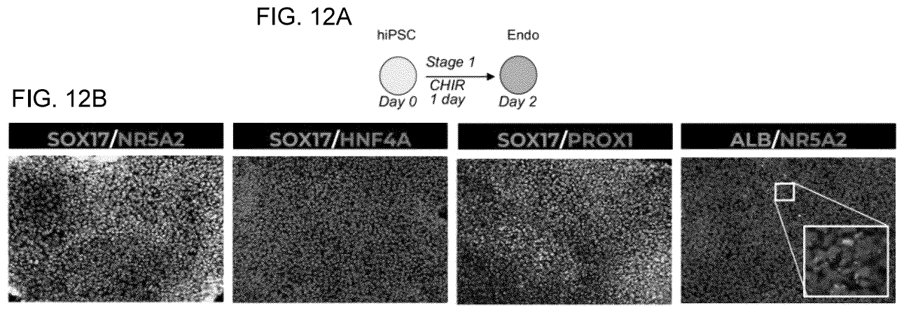

To validate the CHIR-mediated induction of endoderm, an iPSC culture was exposed to CHIR for a single day incubation and then allowed to grow an additional day in basal media. IHC images demonstrated robust co-expression of FOXA2/SOX17, a hallmark of endoderm induction (FIGS. 11A-11B). To assess the hepatic-competence of this endodermal population, a sequential IHC experiment was used to assay for the expression of several early hepatic genes including NR5A2, HNF4A, PROX1, and ALB (FIGS. 12A-12B). While robust expression of NR5A2, HNF4A, and PROX1 was detected, only trace level of ALB was present at day two. Altogether, this suggests that Wnt pathway agonism on an iPSC culture results in an endoderm culture with an inherent hepatic bias.

### Defining Optimal Conditions for an Endoderm Derived Liver Bud

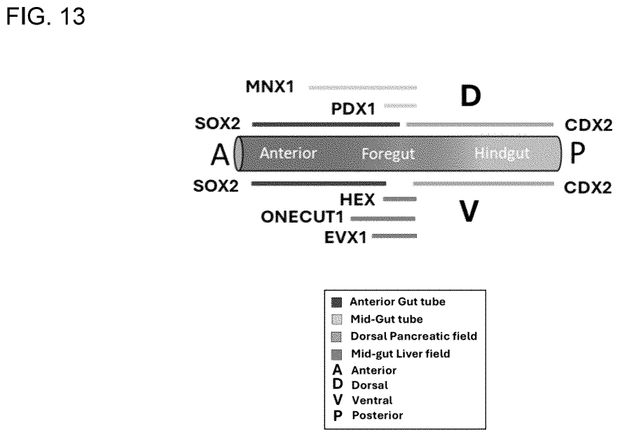

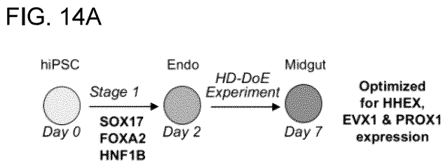

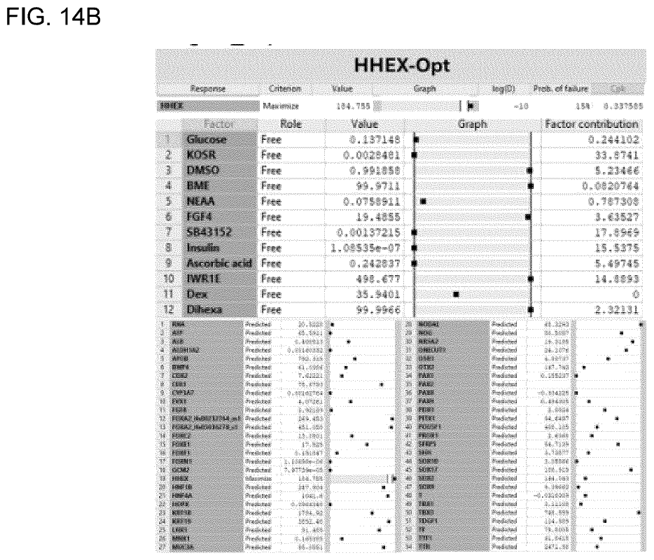

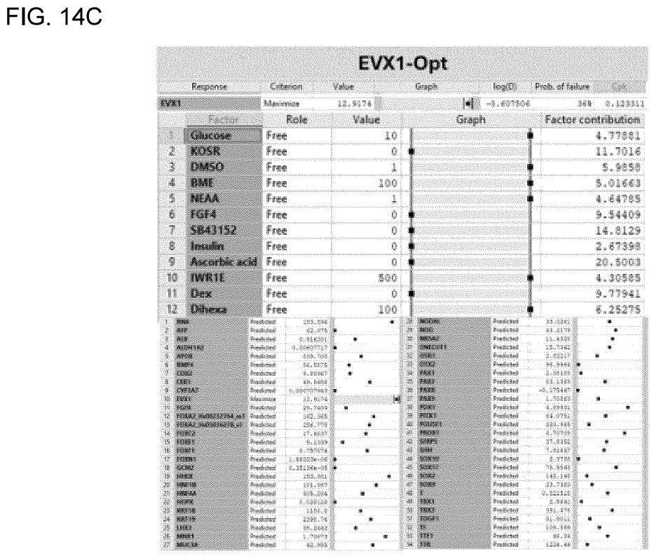

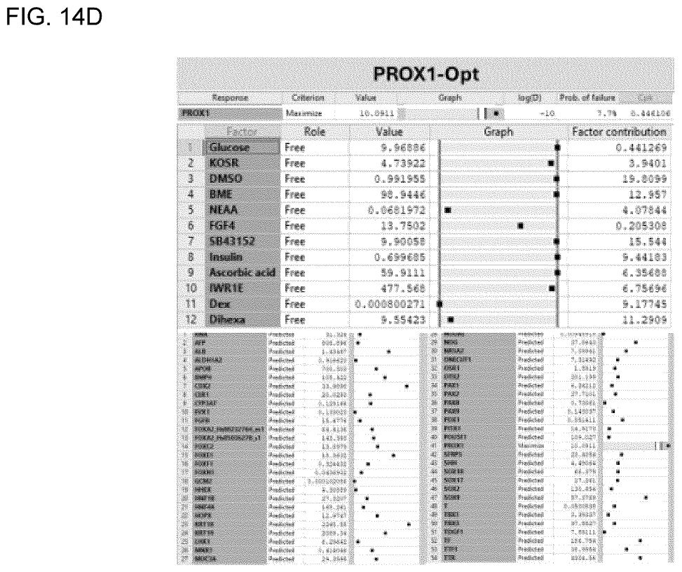

By definition, endoderm is competent to differentiate into any cell of the endoderm lineage. Hepatic bias was detected in the CHIR-mediated induction of endoderm. Thus, sequential modeling experiments were utilized to elucidate how to selectively induce a hepatic fate from the endoderm. A basic understanding of the patterning events that occur across the early gut tube was used to guide this series of modeling experiments. A schematic outlining a simplified expression pattern along the length of the gut-tube is shown (FIG. 13), highlighting the SOX2-expressing anterior region and CDX2-expressing posterior region on either side of the presumptive liver bud region. The midgut region where the liver bud forms is well defined through the co-expression of HHEX, ONECUT1, and EVX1. To establish the optimal conditions needed to induce these three crucial liver-specific midgut genes that are representative of a liver bud, sequential modelling experiments on endoderm cultures were performed. Models for the maximization of HHEX, EVX1, and PROX1 expression were performed (FIGS. 14A-14D) and indicated that DMSO (HDAC inhibitor), BME (a reducing agent commonly used in cell culture), and IWR1E (Wnt pathway inhibitor) were the only compounds within this modeling experiment that contributed to inducing all three genes. The fact that IWR1E was strongly inducing HHEX, EVX1, and PROX1 reinforces the idea that a short, early exposure to Wnt signaling is optimal for liver induction.

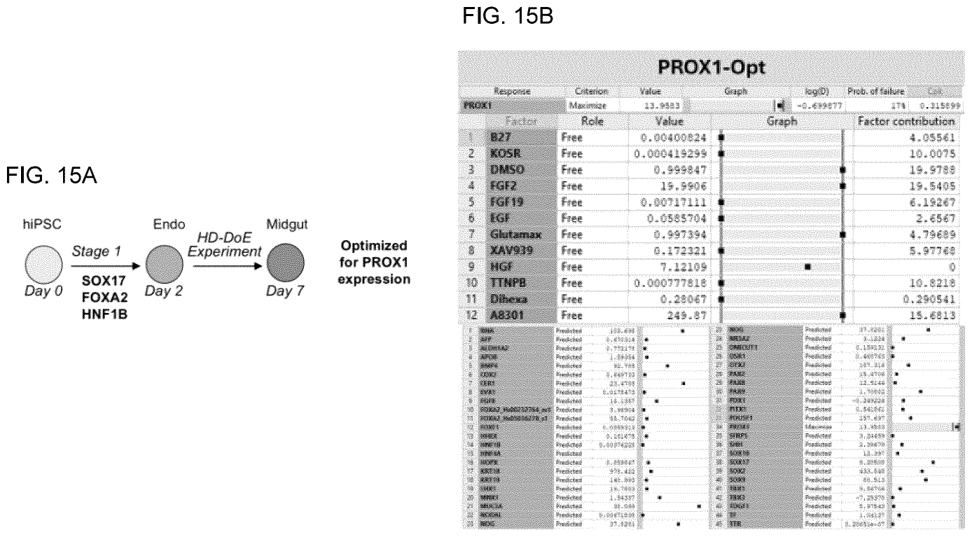

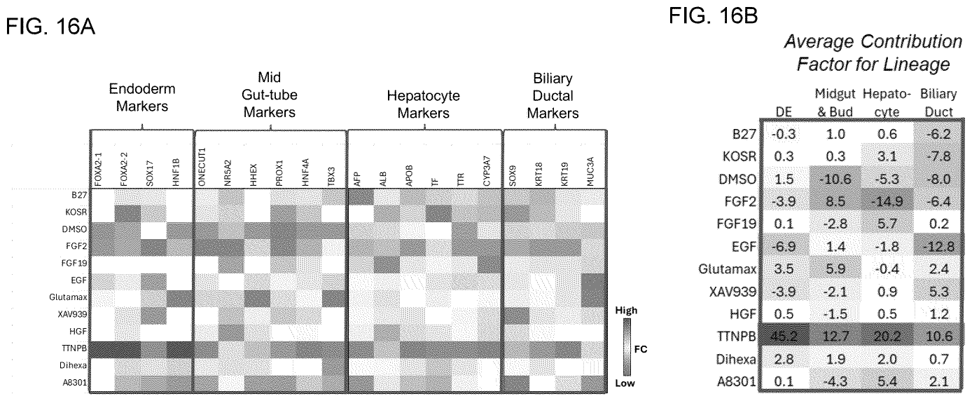

We next focused specifically on the activation of PROX1 to establish the optimal conditions to maximize this gene's expression. An additional modeling experiment established that the optimal activation of PROX1 was accomplished through the combinatorial effects of DMSO, FGF2, and A8301 (FIGS. 15A-15B). This shows that active FGF signaling in the absence of TGF-β signaling and HDAC activity were optimal conditions for the induction of PROX1. Looking more broadly at the activation of midgut genes representative of the liver bud, the effectors'overall contribution to the sequential hepatic lineage inductions was evaluated. This was accomplished by the in silico maximization of genes representative of the different descendent lineages of the liver with their average contribution factors summed. This enabled the evaluation of the relative effector contributions to each discrete stage of the differentiation event (FIGS. 16A-16B). All together this demonstrated a significant contribution of TTNPB, FGF2, and GlutaMAX™ for the induction of the midgut genes with a liver bud phenotype and that active retinoid and FGF pathways contribute towards a hepatocyte fate.

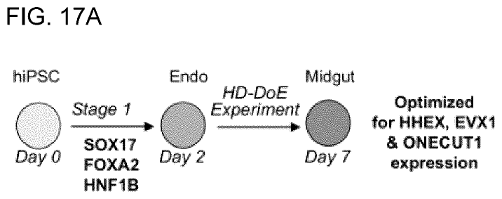

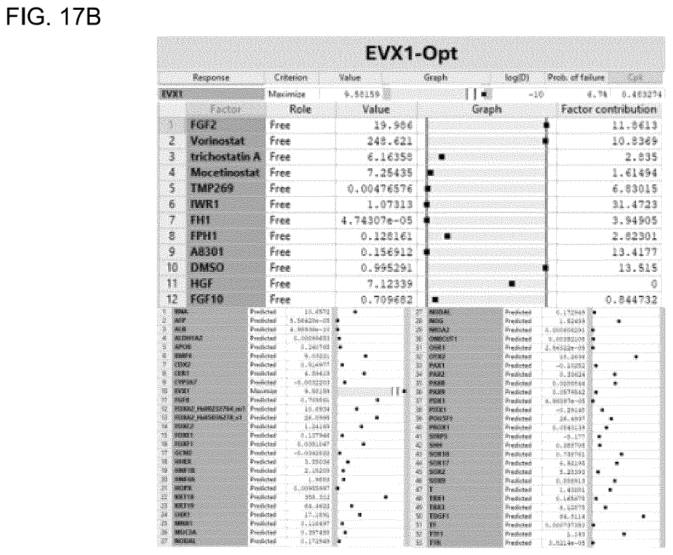

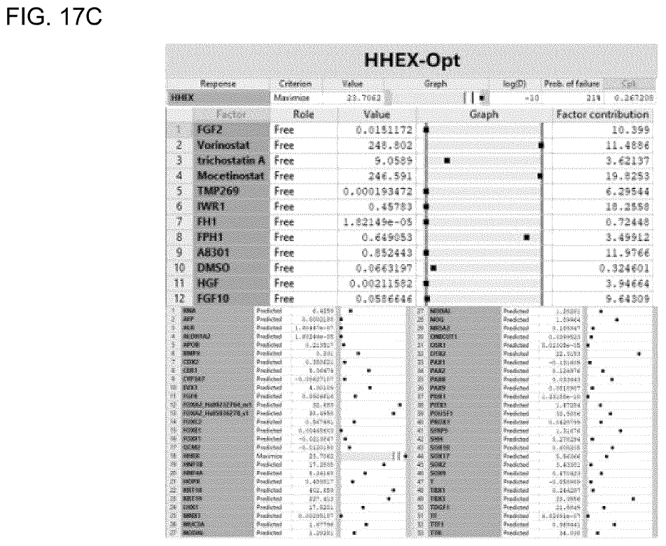

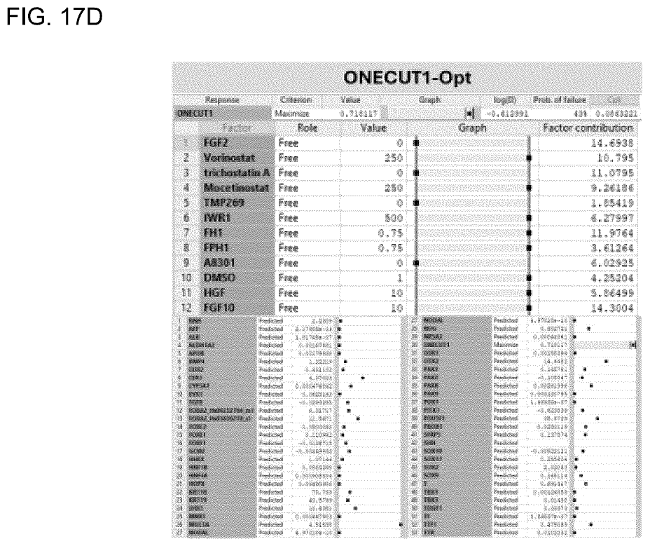

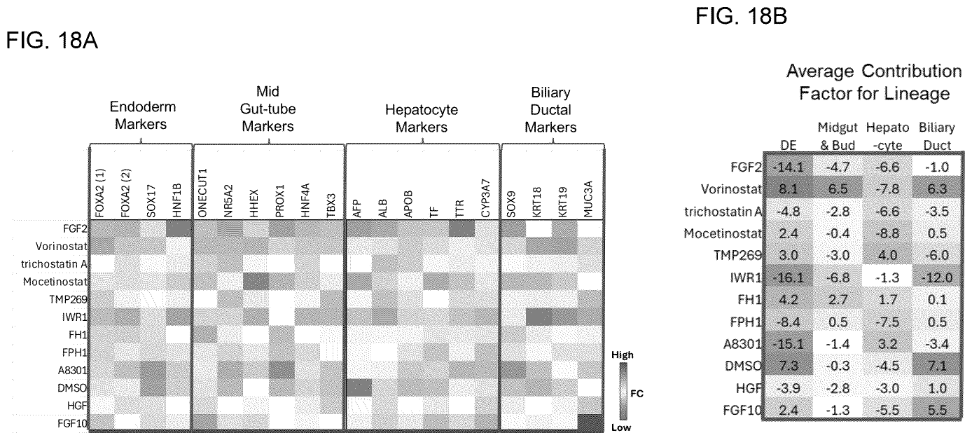

To establish methods for inducing the co-expression of the essential liver bud markers, a sequential HD-DoE modeling experiment with the intent of maximizing EVX1, HHEX, and ONECUT1 induction was performed (FIGS. 17A-17D). The single effector that was universally determined to have a positive inductive effect on these genes was the HDAC inhibitor Vorinostat, while other HDAC inhibitors (DMSO and Mocetinostat) showed varied effects on their induction. Compiling models for the maximization of several hepatic-specific genes representative of the different descendent lineages of the liver confirmed that the only effector that contributed to several genes of the gut-tube region of the liver bud was the HDAC inhibitor Vorinostat, and to lesser extinct FH1 (FIGS. 18A-18B).

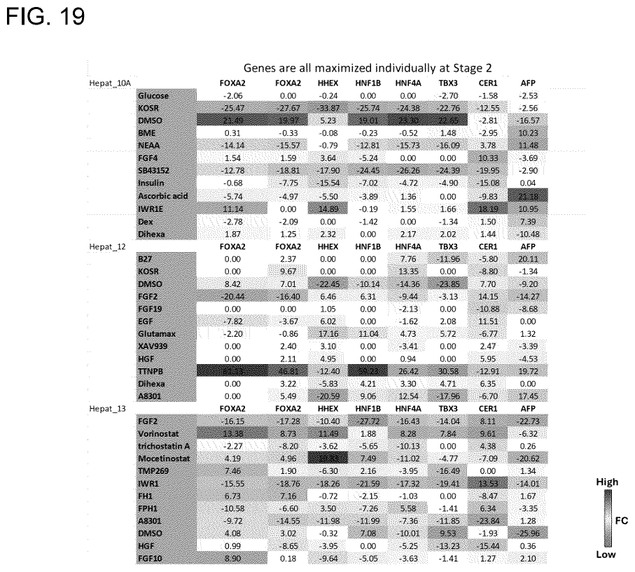

A summary of the factors involved in driving a hepatic fate from endoderm is presented in FIG. 19. To compile the results of multiple HD-DoE experiments, three sequential modeling experiments evaluating 12 factors at a time were combined into a single heatmap. The factors that were evaluated are listed in the left column of the heatmap with the genes being interrogated through optimization for maximum expression listed at the top of the heatmap. The differentiation inducing reagents'impact on the gene induction are listed as FC numbers with larger FC numbers being indicative of the overall strength of the effect. While positive FC numbers represent a positive inducing effect, negative FC numbers represent a negative (inhibitory) effect on the induction of the gene being investigated. This reinforces the previously made observations that DMSO, BME, IWR1E, GlutaMAX™, TTNPB, and Vorinostat show an overall positive effect on the induction of mid-gut genes.

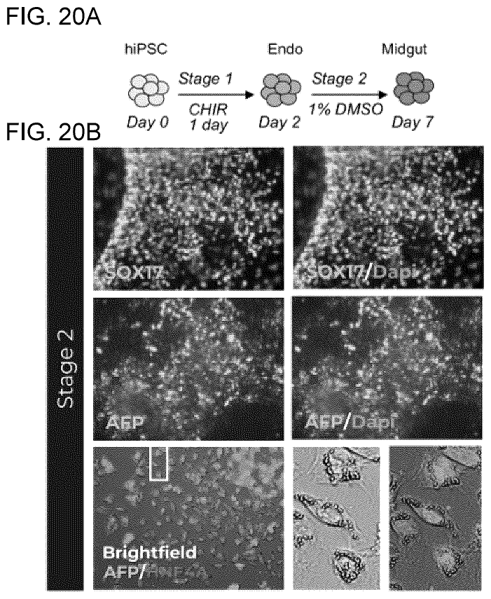

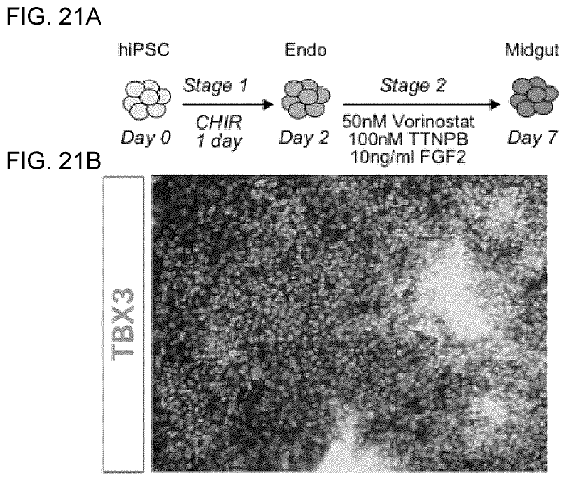

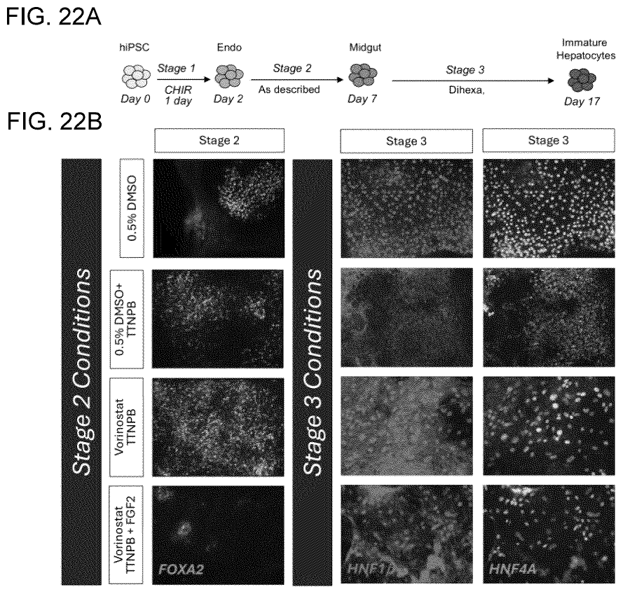

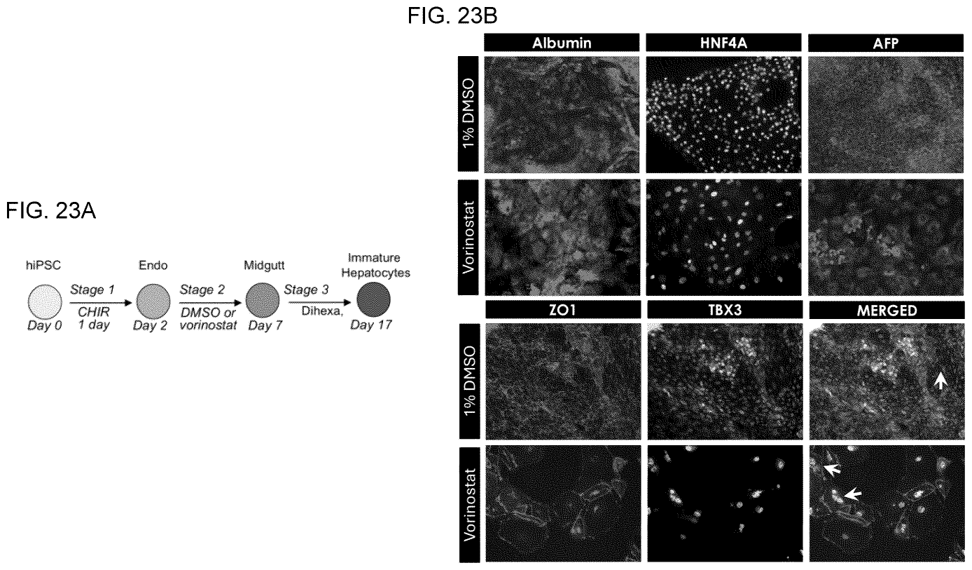

To validate the use of HDAC inhibitors for the induction of midgut, markers representative of the liver bud were assayed using IHC. SOX17, a pan-endodermal marker, was widely expressed in the descendent iPSC-derived midgut population as expected (FIGS. 20A-20B). The hepatic specific genes from this region, AFP and HNF4A, were also shown to be robustly induced under these culture conditions verifying the predictions of the models. To specifically validate the HD-DoE-informed midgut induction method, the models were tested for midgut induction empirically by incubating endoderm culture in the presence of Vorinostat, TTNPB, and FGF2 for five days (FIG. 21A). This resulted in a culture that robustly expressed the midgut marker TBX3 (FIG. 21B). To validate if the HD-DoE-informed midgut induction method had liver competence, Vorinostat, TTNPB, and FGF2-generated midgut was incubated in the presence of an HGF agonist (FIGS. 22A-22B), a method commonly used to induce a hepatic fate from regionalized endoderm. IHC validated that both the midgut (FOXA2 expression) and the descendant hepatic (HNF1B and HNF4A) states achieved a hepatic fate. A similar approach was used to specifically compare the HDAC inhibitor Vorinostat to DMSO (FIGS. 23A-23B). The resulting culture was again incubated in the presence of an HGF agonist to evaluate hepatic competence. Through IHC it was shown that both DMSO and Vorinostat could robustly induce HNF4A/ALB/AFP co-expression, a state representative of the hepatocyte fate.

### Selecting for a Hepatocyte Fate From a Pro-Hepatic Field

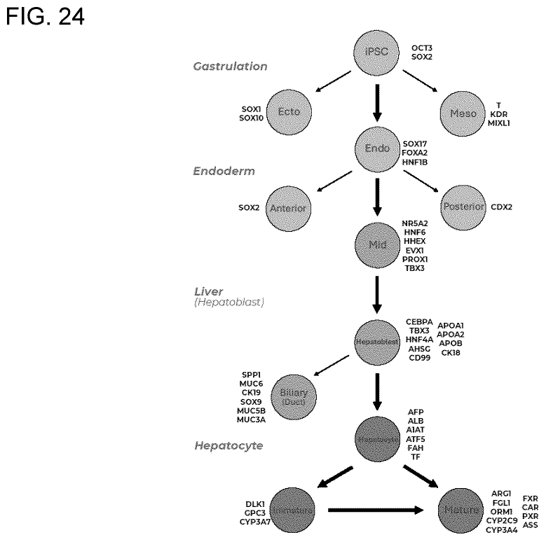

The nascent liver bud field gives rise to two main cell types-the hepatocyte and the cholangiocyte. To properly drive the hepatocyte fate over the cholangiocyte, a set of HD-DoE modelling experiments was used. A schematic showing genes that are differentially expressed throughout the development of different liver-specific fates including the early hepatoblast progenitor, cholangiocytes (ductal cells), and hepatocytes with both an immature and a mature phenotype is shown in FIG. 24. It was determined that the genes that were most important for the initial induction of a hepatocyte fate were AFP, TBX3, HNF4A, and CYP3A7.

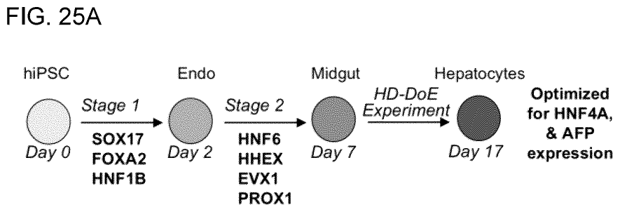

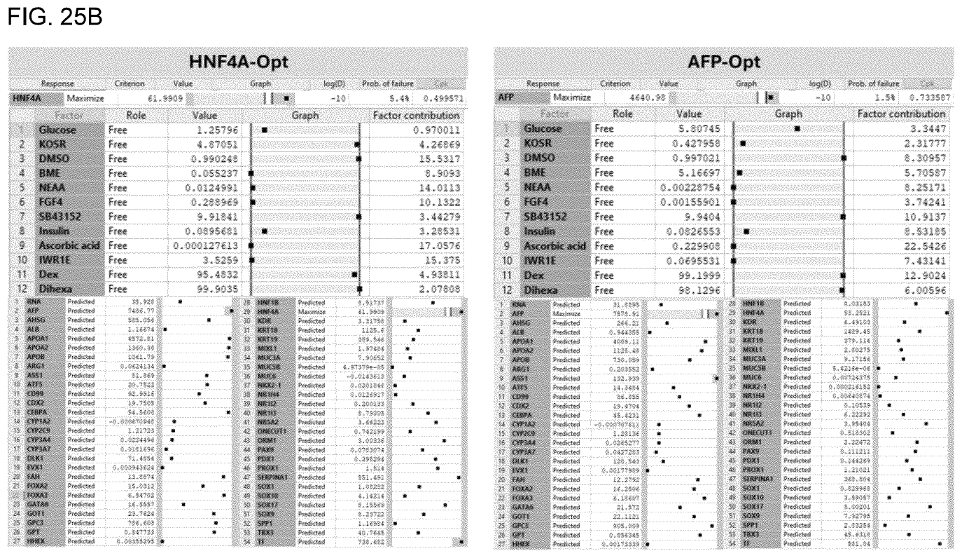

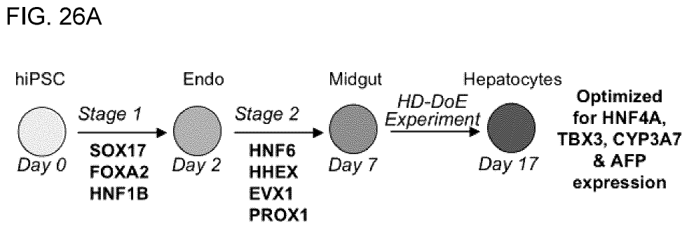

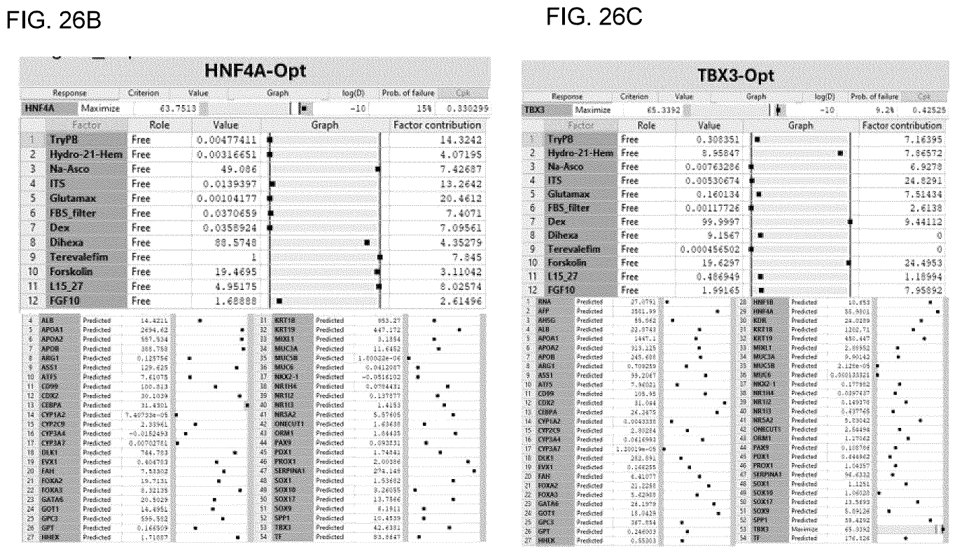

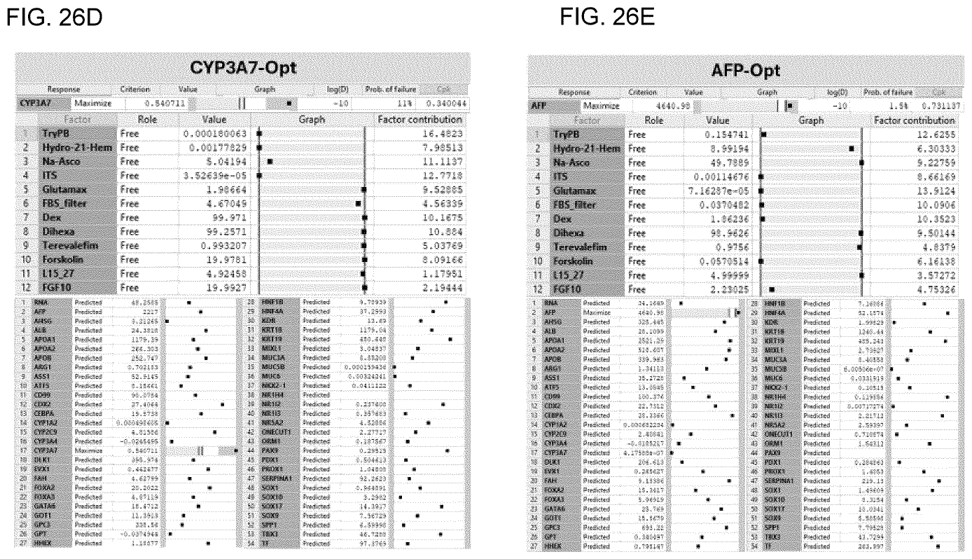

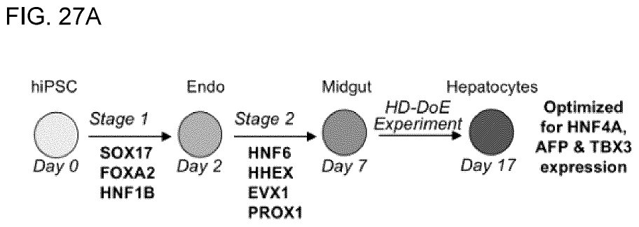

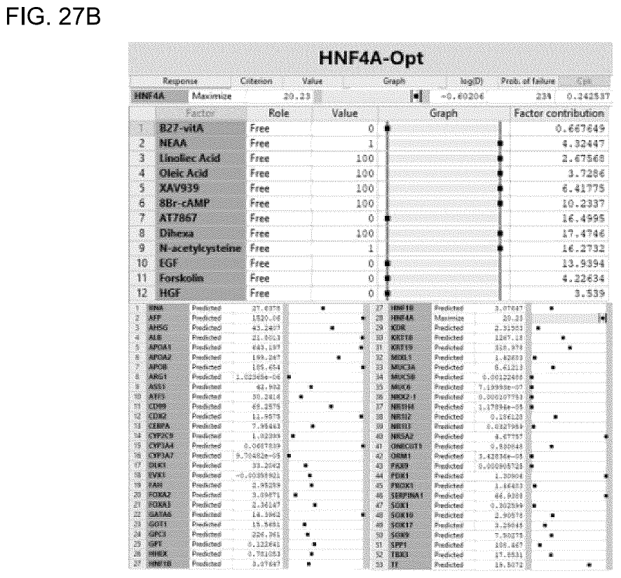

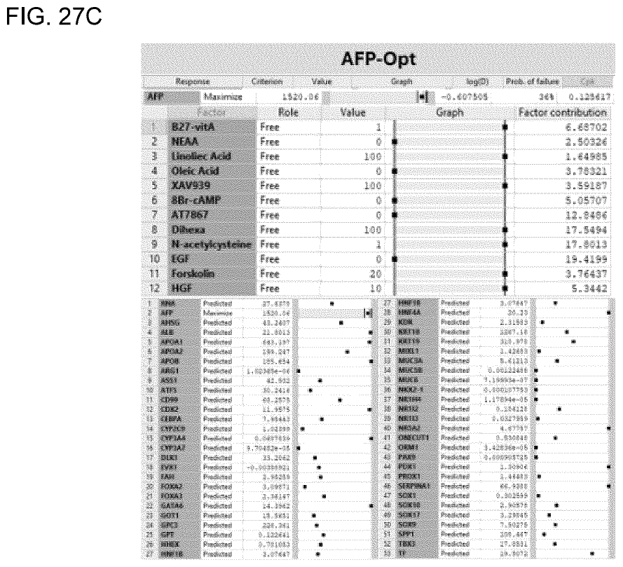

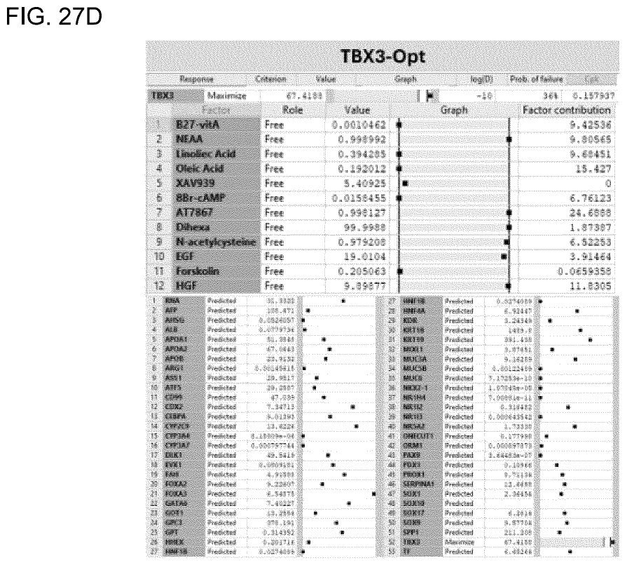

Our initial modeling experiment evaluated the optimal way to induce a hepatocyte from a liver (bud) fated cell culture. The approach taken was to evaluate how to induce the maximal levels of the hepatocyte specific genes, AFP and HNF4A (FIGS. 25A-25B). It was shown that the small molecules DMSO (a HDAC inhibitor), SB431542 (a TGF-β antagonist), Dexamethasone (a glucocorticoid), and Dihexa (an HGF agonist) contributed to the activation of both genes. A subsequent HD-DoE modeling experiment confirmed the importance of HGF signaling pathway agonism and glucocorticoid stimulation in hepatic induction (FIG. 26A). It was shown that Dihexa contributed to the induction of HNF4A, CYP3A7, and AFP, all genes associated with hepatoblasts, though Dihexa showed no effect on TBX3 induction, potentially because TBX3 is already expressed within the midgut population. Dexamethasone was shown to contribute to both TBX3 and CYP3A7 activation. This modeling experiment also established the importance of CREB agonism in hepatic specification, as it strongly contributed to the induction of TBX3 with a contribution factor of 24.5, HNF4A, and CYP3A7 (FIGS. 26B-26E). Additional data supporting the crucial role HGF agonism has in inducing a hepatic fate was shown in a follow-up modeling experiment. Dihexa and HGF were shown to be necessary to maximize the expression of AFP and TBX3, though the models predicted differential effects of the two molecules on the induction of HNF4A, with Dihexa having a significant impact and HGF having little effect on the induction (FIGS. 27A-27D).

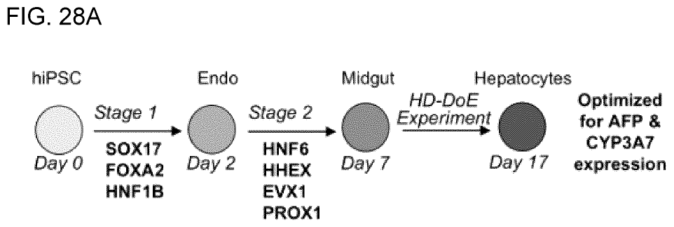

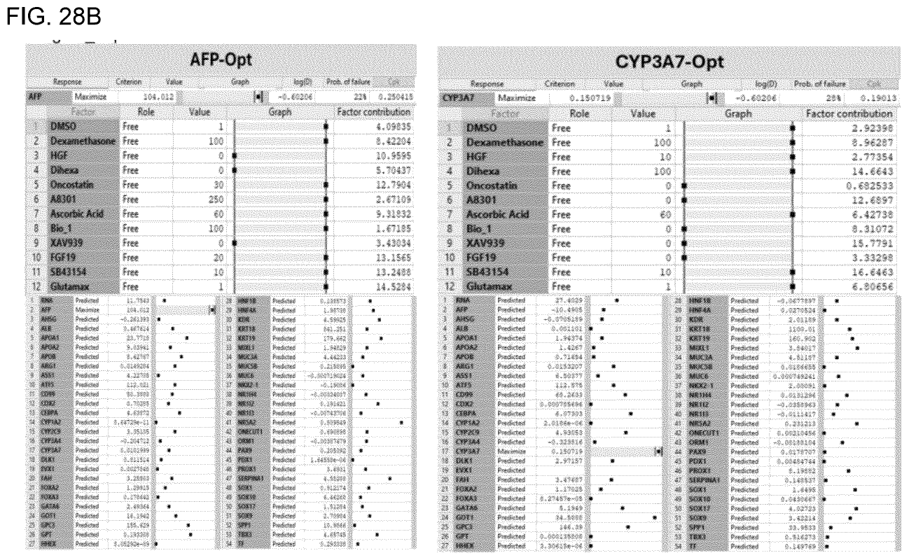

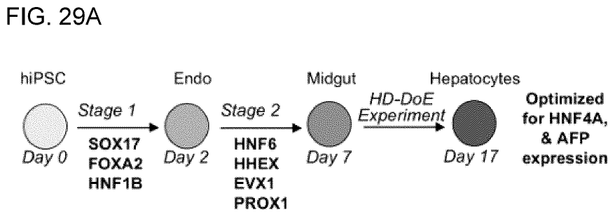

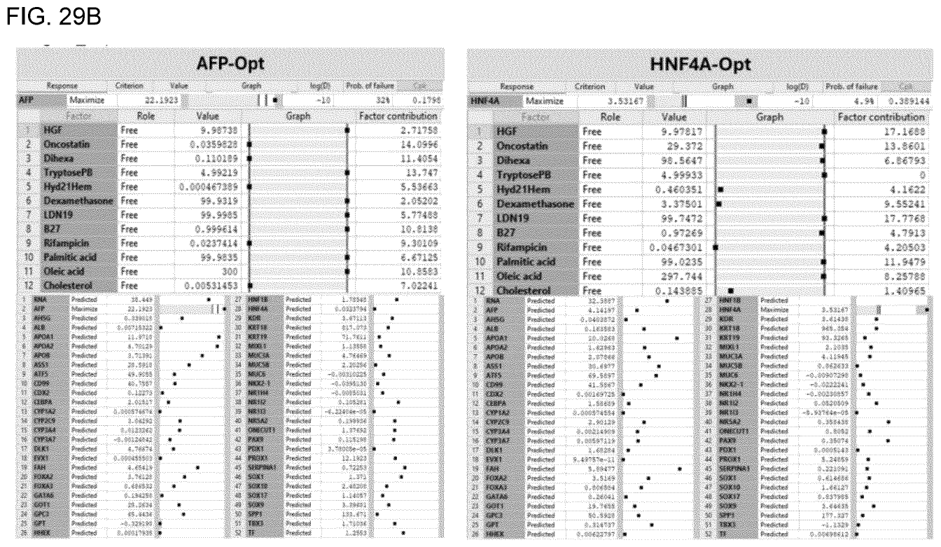

Additional data demonstrating the beneficial effects of the small molecules DMSO (HDAC inhibitor), SB431542 (TGF-β antagonist), and Dexamethasone (glucocorticoid) on the induction of a hepatic fate is shown (FIGS. 28A-28B). Optimizing for the maximal expression of AFP and CYP3A7 identified all these molecules as contributing to their expression. In addition, both ascorbic acid and GlutaMAX™ were also identified as effectors that positively contributed to the induction of AFP and CYP3A7 (FIGS. 28A-28B). A final modeling experiment was performed evaluating how to maximize the induction of the two hepatic genes AFP and HNF4A (FIGS. 29A-29B). Through this experiment it was shown again that HGF agonism had an overall positive inductive effect on hepatic differentiation. In addition, it was shown that inhibition of the BMP pathway, through use of LDN193189, and the presence of two fatty acids, palmitic and oleic acids, positively contributed to the induction of AFP and HNF4A. In addition, the commonly used cell culture additives tryptose and B27 increased expression levels of AFP and HNF4A (FIG. 29B).

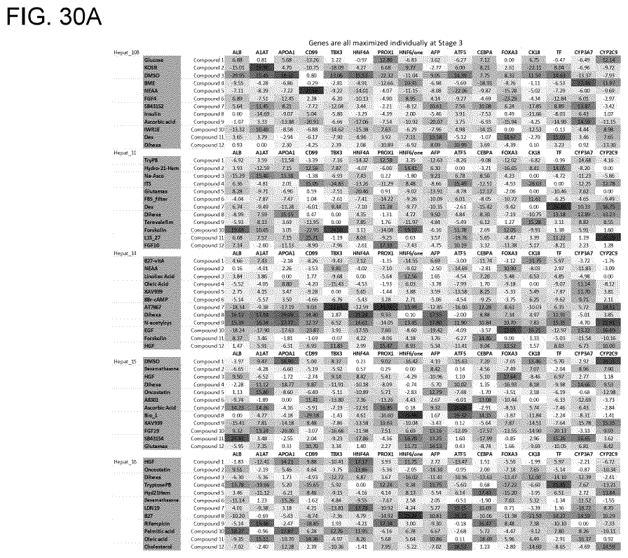

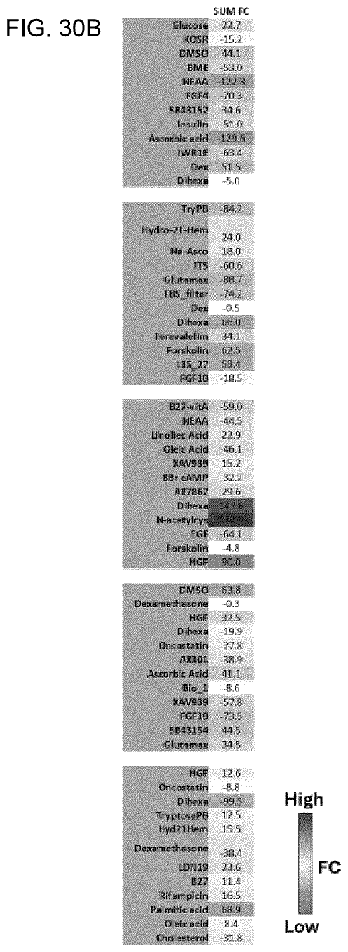

To compile the results of multiple HD-DoE experiments, the five sequential modeling experiments evaluating 12 factors at a time were combined into a single heatmap (FIG. 30A). The factors that were evaluated are listed in the left column of the heatmap, with the genes being interrogated through optimization for maximum expression listed at the top of the heatmap. The genes integrated all represented genes that should be expressed within a hepatoblast or the early budding liver. The differentiation-inducing reagents'impact on the gene induction is listed as FC numbers with larger FC numbers being indicative of the overall strength of the effect. While positive FC numbers represent a positive inducing effect, negative FC numbers represent a negative (inhibitory) effect on the induction of the gene being investigated. All FC numbers from all the genes presented were summed to evaluate their overall contribution to hepatic induction. The effectors throughout this series of experiments that demonstrated a positive contribution for inducing a liver fate rank ordered from the highest contribution to the lowest contribution are: N-acetylcysteine, Dihexa, HGF, palmitic acid, DMSO, forskolin, dexamethasone, SB431542, ascorbic acid, GlutaMAX™, terevalefim, AT7867, hydro-21hem, LDN193189, linoleic acid, glucose, sodium ascorbate, rifampicin, XAV939, tryptose, B27, and oleic acid (FIG. 30B).

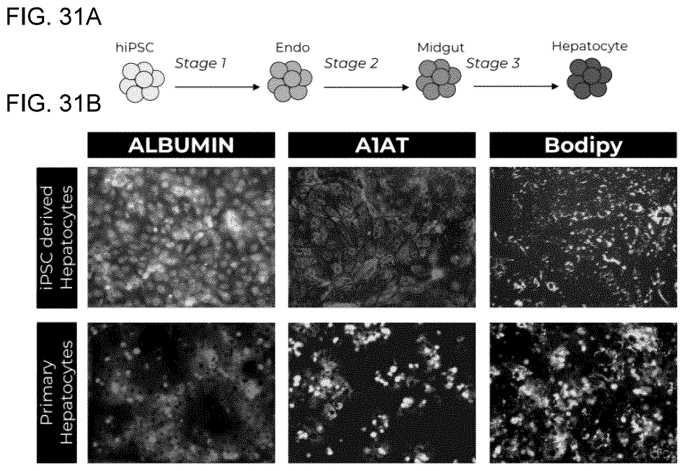

To evaluate the hepatocytes that are being produced throughout this method, the iPSC-derived hepatocytes were compared to primary hepatocytes (FIGS. 31A-31B). Albumin production was observed in the iPSC-derived hepatocytes. Albumin is the most abundantly produced and secreted protein of the hepatocyte. In comparison to the primary hepatocytes, the production of Alpha 1 anti-trypsin (A1AT) was also observed. A1AT deficiency is a primary driver of COPD. Another primary function of hepatocytes is lipid storage. Using Bodipy, lipid droplets were detected in both iPSC-derived hepatocytes as well as the primary hepatocytes. Characterizing the iPSC-derived hepatocytes showed that they retained TBX3expression, a marker commonly associated with a fetal hepatocyte (FIGS. 32A-32B). However, when evaluating primary hepatocytes, TBX3 expression was also retained, suggesting the regenerative properties of hepatocytes. As expected, when iPSC-derived hepatocyte aggregates were plated, they showed a clear epithelial phenotype as evident through ZO1 expression. Further, iPSC-derived hepatocytes maintained the epithelial morphology after digestion. The low expression of CYP3A4 demonstrated an immature phenotype of the iPSC-derived hepatocytes at this stage. CYP3A4 is a commonly accepted maturation marker, and further optimization for CYP3A4 expression should improve the maturation level of the cells.

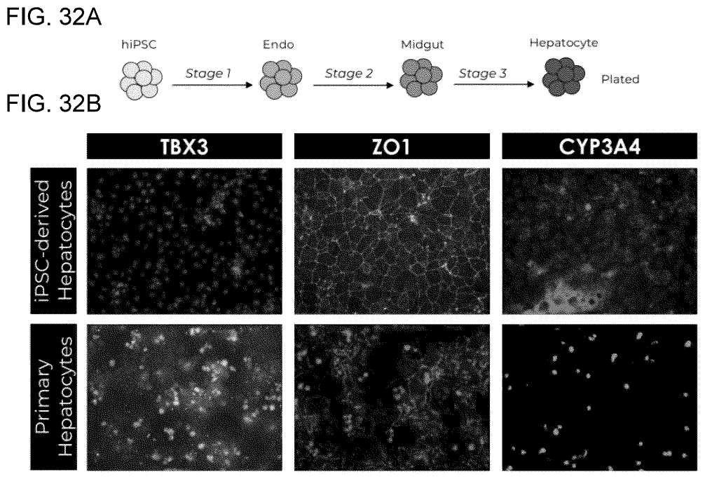

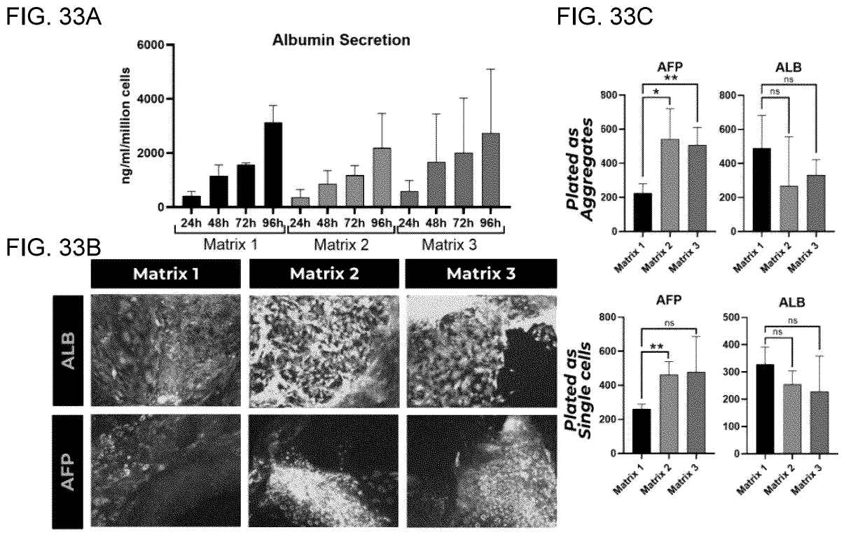

To evaluate the hepatocyte on a functional level their ability to secrete albumin was investigated (FIGS. 33A-33C). Bioreactor generated iPSC-derived Hepatocytes were plated onto three different ECM molecules and evaluated for albumin secretion. It was determined that the iPSC-derived hepatocytes secrete ˜453 ng/day/million (FIG. 33A), an approximate ˜30% secretion level of primary hepatocytes which are reported to secrete albumin at a rate of 1.5 ug/day/million cells when in culture. No statistical difference in albumin expression or secretion was detected on any of the different ECM substrates tested. However, a statistically significant decrease in AFP expression could be achieved depending on what ECM molecules are present (FIG. 33C). Decreased AFP expression is a hallmark of a more mature hepatocyte.

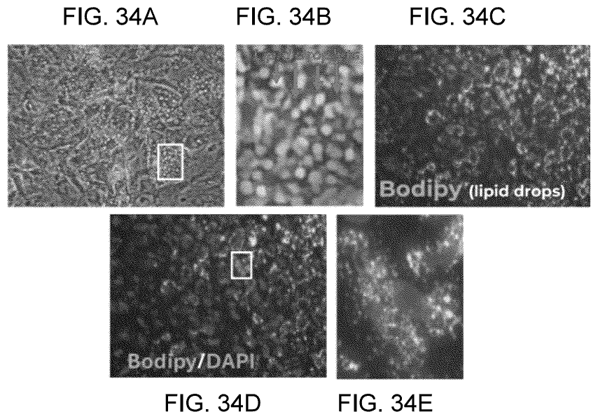

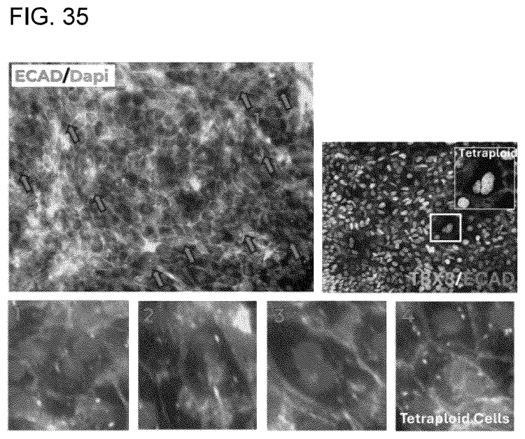

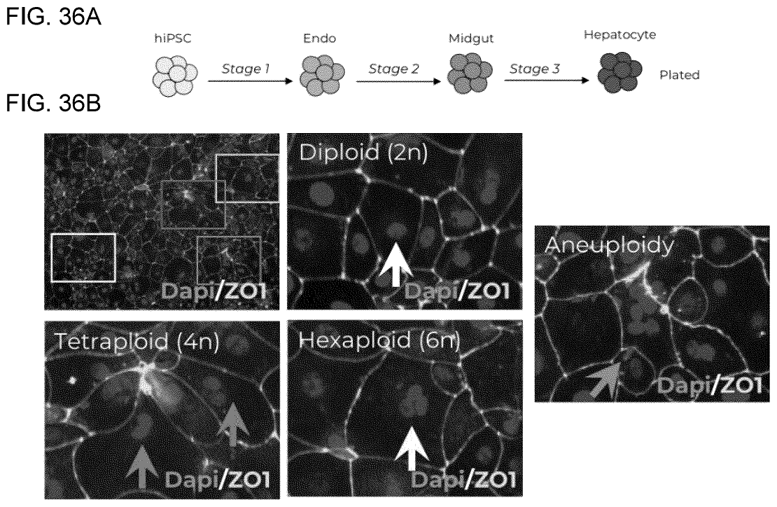

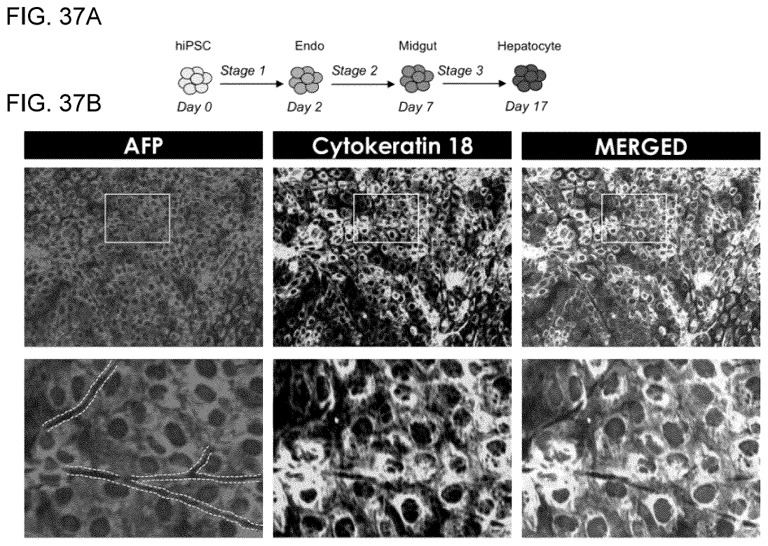

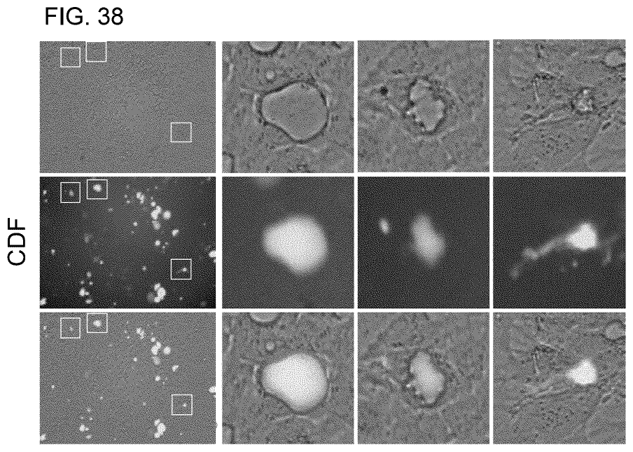

Further characterization of the iPSC-derived hepatocytes focused on the occurrence of lipid drop accumulation through close examination of brightfield images of the iPSC-derived hepatocytes (FIG. 34A). Lipid drops are clearly visible and are indicated using arrows (FIG. 34B). In addition, the dye Bodipy retained within lipid droplets outline the accumulated droplets (FIGS. 34C-34E). There is widespread accumulation of this dye within the iPSC-derivatives. An additional quality of hepatocytes is that they are polyploidy in nature. This feature was clearly demonstrated in the iPSC-derived hepatocytes, as the occurrence of polyploidy cells were commonly found (FIG. 35). Hepatocytes are binucleated at birth and increase throughout post-natal development as the polyploidies of cells become greater in number as they mature. Approximately 30% of hepatocytes in adult liver have some degree of polyploidy. Polyploid hepatocytes occur up to 16 n per cell and occur through incomplete cytokinesis, though cellular fusion may also be involved. As an individual ages, the number of polyploid cells increases, and there is some implication in chronic liver diseases. The iPSC-derived hepatocytes show several levels of polyploidy as indicated in FIGS. 36A-36B. Occurrences of cells containing between two and five nuclei per cell have been observed (FIG. 36B). Another feature of the iPSC-derived hepatocytes is that they form canaliculi structures (FIGS. 37A-37B). Canaliculus structures function for the selective secretion between the hepatocytes and neighboring cells. Canaliculi are spaces formed between the adjacent hepatocytes. Secretion of hepatic bile has a directional flow into the bile ducts via canaliculi. The white dotted line outlines canaliculus areas (FIG. 37B). These canaliculi were found to be functional, as evidenced by transport of CDF from iPSC-derived hepatocytes to pseudo-canaliculus areas (FIG. 38).

### Increasing iPSC-Derived Hepatocytes Functionality Through Targeting CYP Activation

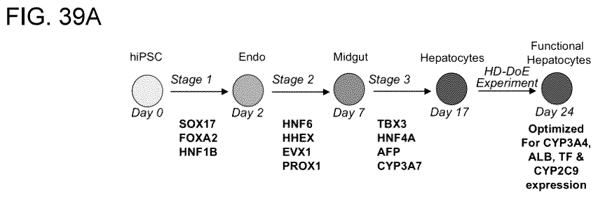

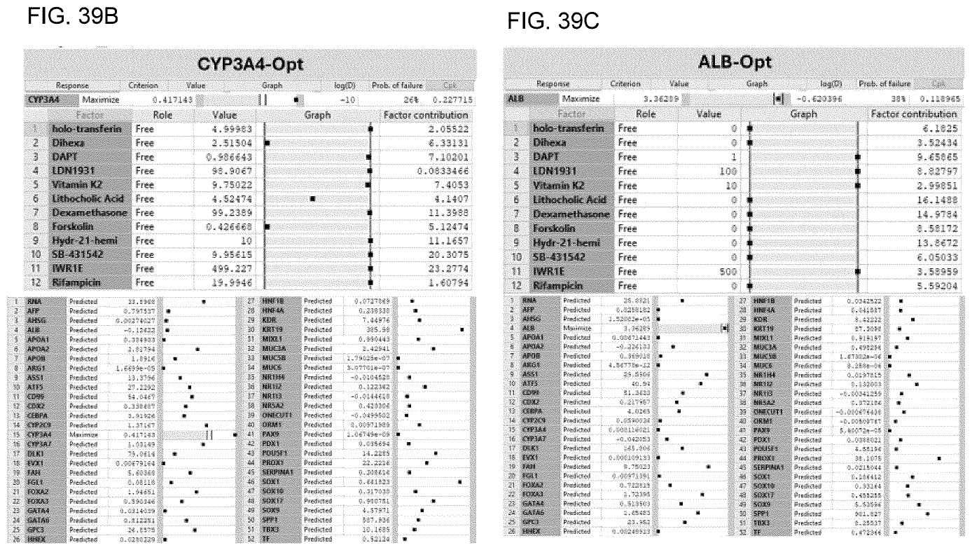

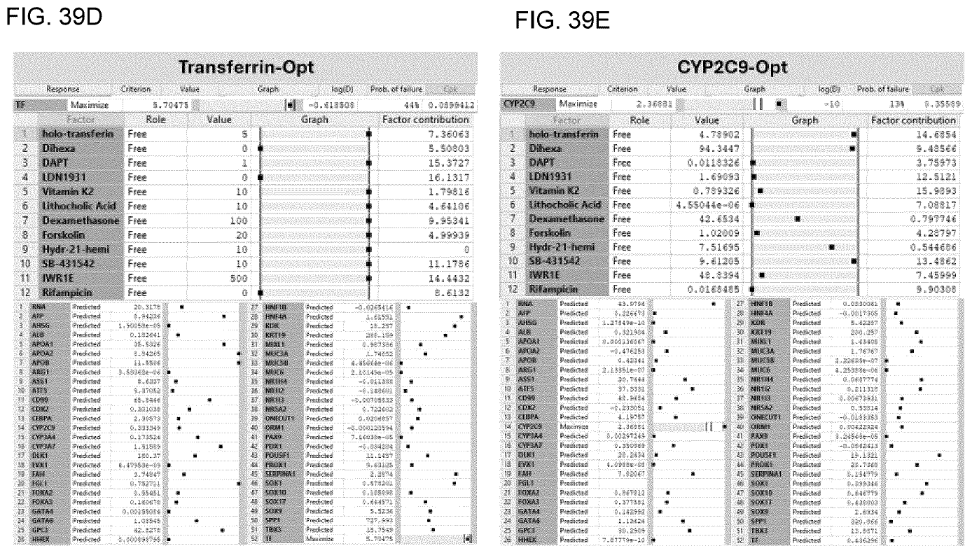

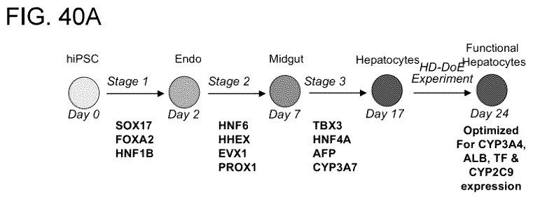

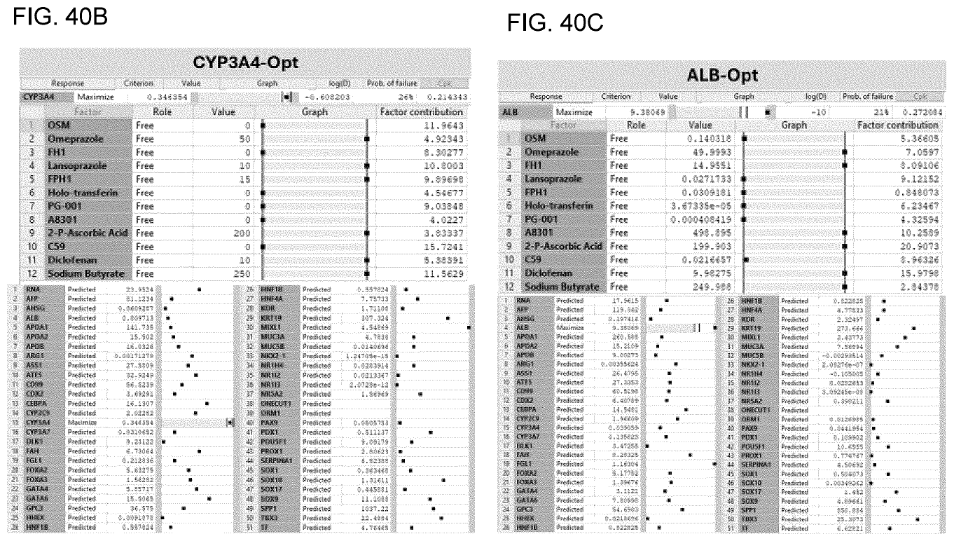

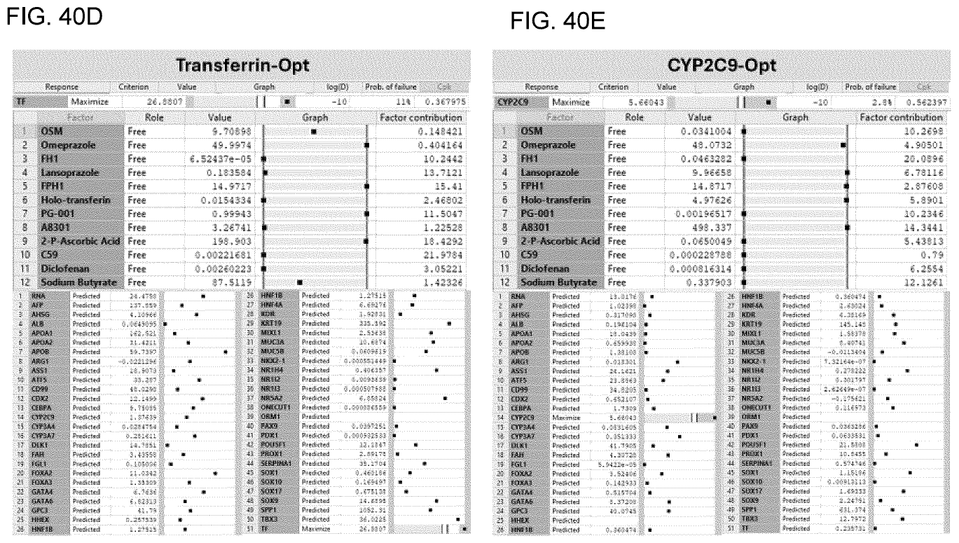

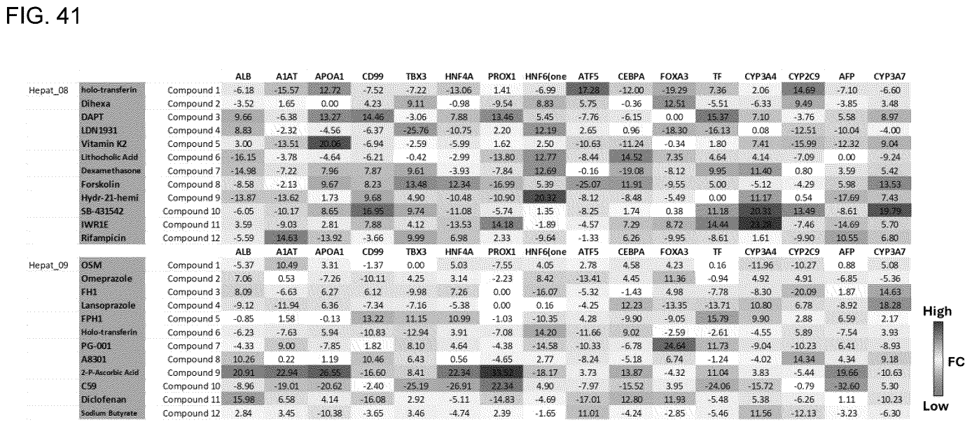

An HD-DoE approach was used to evaluate the optimal way to induce a functional state within the iPSC-derived hepatocytes. The initial steps were evaluating how to induce the maximal levels of the functional-related genes CYP3A4, ALB, TF, and CYP2C9 (FIGS. 39A-39E). It was determined through maximization of these genes that vitamin K2 (a microbial derived metabolite) and IWRIE (a Wnt pathway inhibitor) increased the activation of all genes assayed. Dexamethasone and SB431542 both increased the induction of TF, CYP2C9, and CYP3A4, but not ALB. A subsequent modeling experiment determined that omeprazole increased induction of CYP3A4, ALB, TF, and CYP2C9 (FIGS. 40A-40E). While FPH1increased the expression of TF, CYP3A4, and CYP2C9, 2-P-ascorbic acid and sodium butyrate both increased the expression of CYP3A4, ALB, and TF, but not CYP2C9. To take a broader approach, optimization for the maximal expression of mature hepatocyte genes was measured through this series of experiments. To increase the functional level of the iPSC-derived hepatocytes, additional HD-DoE modeling experiments were used. All of the genes measured that are associated with mature hepatocytes were optimized individually and the representative contribution factors for the components evaluated are shown (FIG. 41).

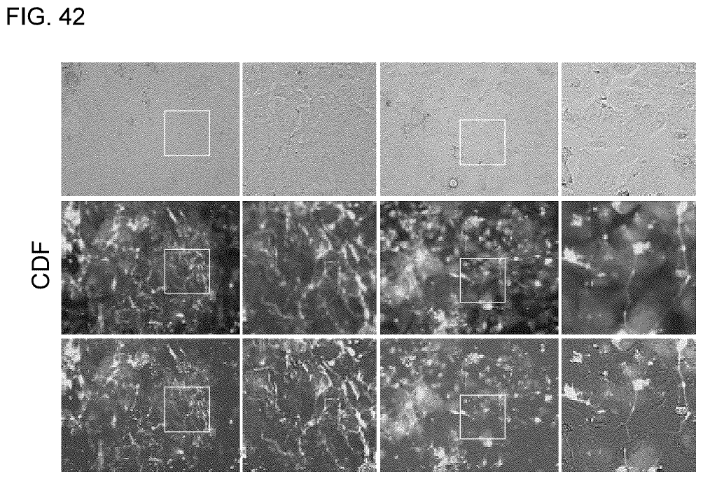

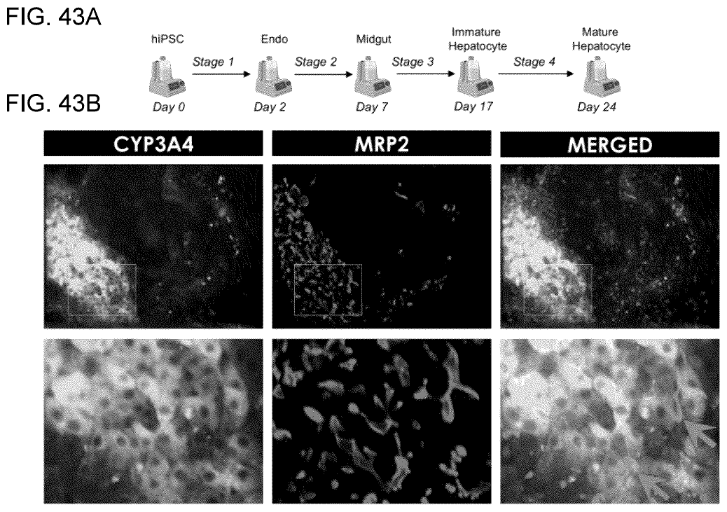

Stage 4 optimization retained the ability to form functional hepatocyte canaliculus structures (FIG. 42). The iPSC-derived hepatocytes expressed maturation markers which were associated with cell density. Additionally, when hepatocytes were grown in confluent cultures, they displayed polarity. The CYP450-associated gene CYP3A4 expression was associated with cell density. CYP3A4 and MRP2 were co-expressed (FIGS. 43A-43B). MRP2 is an apical hepatocyte marker that is present within the canaliculus. Bile canaliculus structures were observed in the aggregate derived hepatocyte.

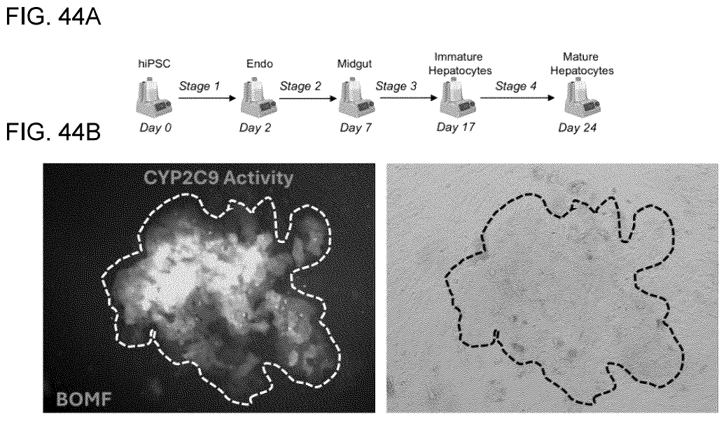

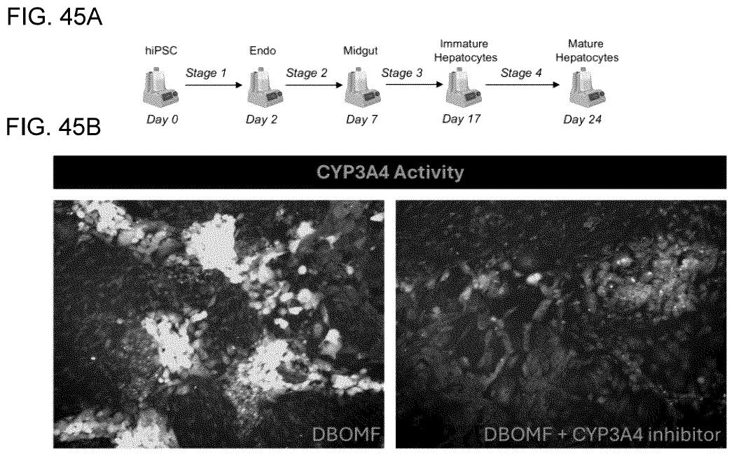

Stage 4 cells also displayed CYP450 activity, a critical attribute in toxicity screens. CYP450 is a class of liver enzymes used in the detoxification of blood. CYP2C9 and CYP3A4 are markers of a mature hepatocyte, and their activities were measured using cells that were grown in bioreactors, cryopreserved, and recovered. Cells were exposed to chromogenic reagents, and a clear florescent signal was detected for CYP2C9 activity (FIGS. 44A-B). CYP3A4 activity was also validated through parallel reactions in which cells were exposed to a specific chromogenic reagent in the absence and presence of a CYP3A4 inhibitor ketoconazole (FIGS. 45A-B). CYP3A4 is involved in the detoxification of about 50% of all medicine.

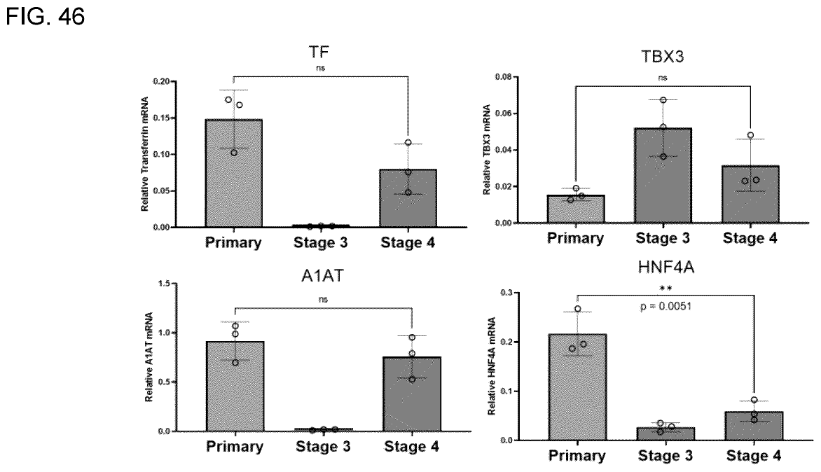

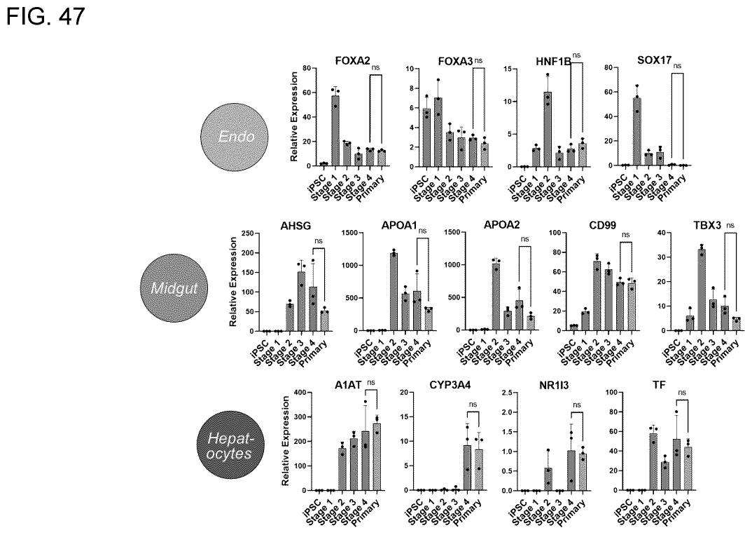

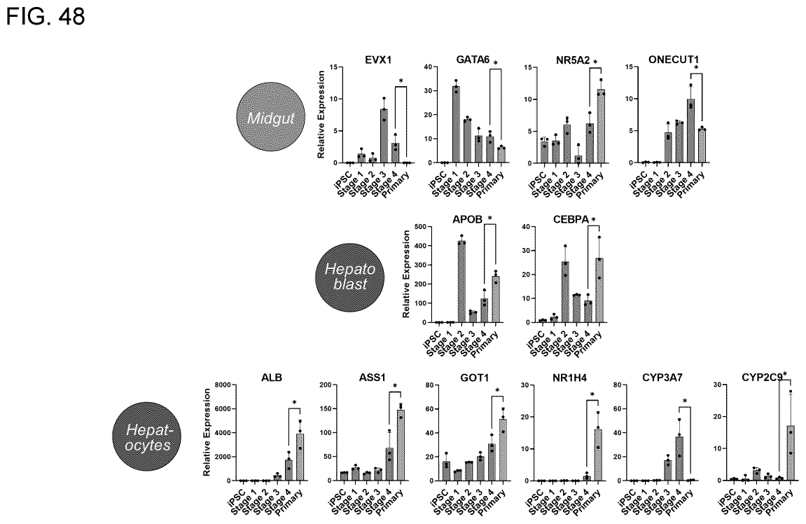

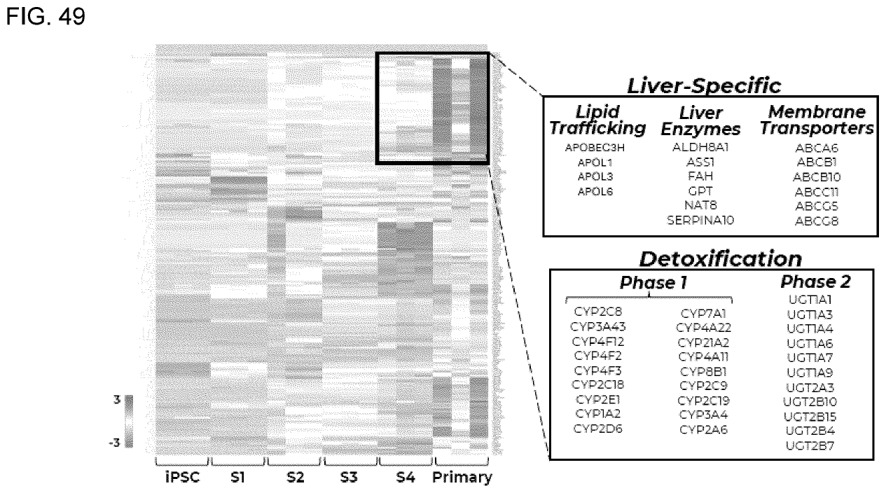

To further validate the increased mature state of Stage 4 cells, RNA-based evaluation of the transition from stage 3 to stage 4 cultures compared to primary hepatocytes was performed (FIG. 46). It was shown that through incubation in the presence of stage 4 media for a week that the iPSC-derived hepatocytes had transcript levels of TF and A1AT similar to primary hepatocytes with no significant differences between the two populations of cells (FIG. 46). A subsequent stagewise assessment of hepatocyte induction protocol demonstrated the progression to a more mature state (FIG. 47). The serial expression patterns of the stagewise progression of the iPSC-derived hepatocytes were directly compared to primary hepatocytes. It was shown that there were no significant differences in the levels of expression between the stage 4 cells and primary hepatocytes for the general endoderm genes FOXA2, FOXA3, HNF1b, and SOX17, the midgut genes AHSG, APOA1, APOA2, CD99, and TBX3, and the hepatocyte specific genes A1AT, CYP3A4, NR113, and TF (FIG. 47). In addition, little significant difference between the stage 4 and primary hepatocyte was seen in the expression levels for the midgut genes EVX1, GATA6, NR5A2, and ONECUT1 (HNF6), the hepatoblast genes APOB and CEBPA, and the hepatocyte specific genes ALB, ASS1, GOT1, NR1H4, CYP3A7, and CYP2C9 (FIG. 48). RNA-based evaluation of the various cell populations obtained throughout the differentiation protocol is shown in FIG. 49.

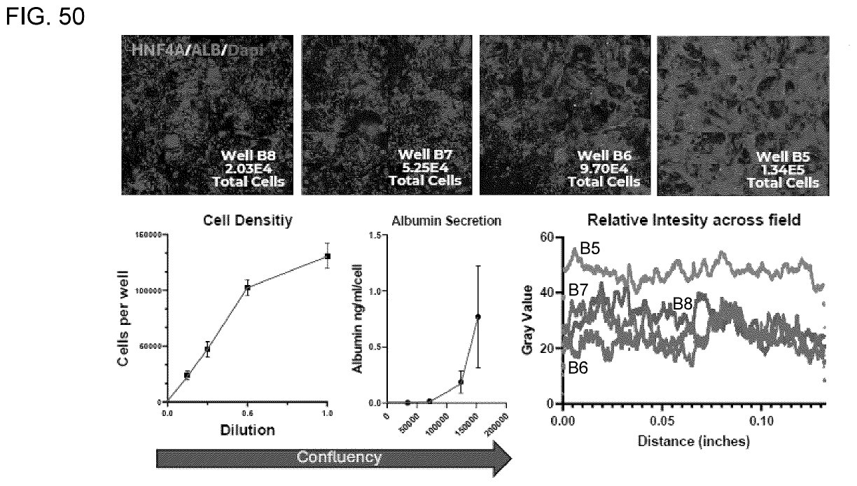

To examine the effects of cellular density on hepatic albumin expression, cells were seeded at different densities (2.03×10⁴, 5.25×10⁴, 9.70×10⁴, and 1.34×10⁴), incubated at 37° C. for 5 days, and evaluated for albumin staining and secretion. As cultures approached 100% confluency, the intensity of albumin staining and the amount of albumin secretion increased (FIG. 50). Similar patterns of increased expression were observed with HNF4A and CYP3A4 (data not shown).

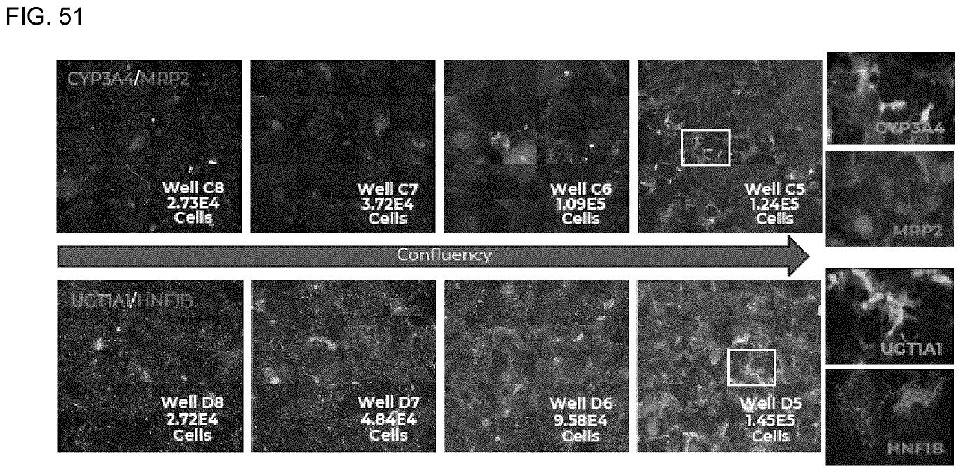

Lastly, to determine whether cellular density affects mature hepatocyte expression, cells were seeded at different densities (2.72×10⁴, 3.72×10⁴, 1.09×10⁵, and 1.24×10⁵), incubated at 37° C. for 5 days, and evaluated for CYP3A4, MRP2, UGT1A1, and HNF1B. As cultures approached 100% confluency, the expression of CYP3A4 and UGT1 A1 increased (FIG. 51).

Those skilled in the art will recognize or be able to ascertain, using no more than routine experimentation, many equivalents of the specific embodiments of the invention described herein. Such equivalents are intended to be encompassed by the following claims.

1. A method of generating a population of hepatocytes from induced pluripotent stem cells (iPSCs) comprising:
(a) culturing iPSCs in a culture medium comprising a glycogen synthase kinase 3 beta (GSK3B) inhibitor for 12-120 hours to obtain a first population of endodermal cells;
(b) culturing the first population of endodermal cells from step (a) in a culture medium lacking a GSK33 inhibitor for 12-120 hours to obtain a second population of endodermal cells;
(c) culturing the second population of endodermal cells from step (b) in a culture medium comprising a histone deacetylase (HDAC) inhibitor, a retinoic acid (RA) agonist, and a fibroblast growth factor receptor (FGFR; e.g., FGFR1) agonist for 24-240 hours to obtain a population of midgut cells;
(d) culturing the population of midgut cells in a culture medium comprising a glucocorticoid, a cAMP response element-binding protein (CREB) agonist, an antioxidant, a Wnt antagonist, a transforming growth factor beta (TGF-β) antagonist, and a hepatocyte growth factor (HGF) agonist for at least 48 hours to obtain a population of hepatoblasts; and
(e) culturing the population of hepatoblasts in a culture medium comprising an HDAC inhibitor, a glucocorticoid, a CREB agonist, an antioxidant, a Wnt antagonist, a TGF-β antagonist, an HGF agonist, a Notch antagonist, a bone morphogenetic protein (BMP) antagonist, and a hepatocyte function enhancer for at least 48 hours to obtain a population of hepatocytes.

2. The method of claim 1, wherein:
(a) the GSK36 inhibitor is selected from the group consisting of CHIR99021, CHIR98014, CHIR98023, 3F8, A 1070722, AR-A014418, BIO, BIO-acetoxime, 6-BIO, Indirubin-3′-oxime, Alsterpaullone, 1-Azakenpaullone, Cazpaullone, Kenpaullone, Aloisine A, SB 216763, SB 415286, SB41528, SAR502250, TC-G 24, TWS119, LY2090314, AT7519, KY19382, AZD1080, AZD2858, Hymenialdisine, Debromohymenialdisine, Dibromocantherelline, Meridianine A, NSC 693868, IM-12, IMID1, IMID2, VP2.51, VP2.54, BIP-135, JGK-263, MMBO, TCS2002, PF-367, BRD0705, BRD3731, AF3581, TDZD-8, NP00111, Tideglusib (NP031112), NP031115, L803, L803-mts, L807-mts, HMK-32, Palinurin, Tricantin, Manzamine A, BTO, VP0.7, VP1.14, VP1.16, VP3.15, VP3.35, SC100, 6j, LCQFGS01, LCQFGS02, 4-3, and 4-4;
(b) the HDAC inhibitor in each of steps (c) and (e) is independently selected from the group consisting of Vorinostat, Sodium Butyrate, Belinostat, Dacinostat, Droxinostat, Domatinostat, Entinostat, Ricolinostat, Resminostat, Abexinostat, Givinostat, Ivaltinostat, Panobinostat, Pracinostat, Quisinostat, Mocetinostat, Nanatinostat, Nexturastat A, Tefinostat, Tucidinostat, Fimepinostat, Citarinostat, Zabadinostat, GSK3117391, CUDC-101, AR-42, M344, Scriptaid, Sulforaphane, SR-4370, MC1568, CAY10603, CAY10683, RG2833, RGFP966, Cpd60, BRD3308, Tasquinimod, BML-210, LMK-235, BRD73954, PCI-34051, TMP195, TMP269, NKL22, TH34, SIS17, WT-161, ACY-738, BG45, CBHA, Pyroxamide, NCH-51, NCH-31, KD 5170, TCS HDAC6 20b, NSC 3852, NSC 69603, NSC 86371, NSC 305819, MS-27-275, Trapoxin A, Trapoxin B, Romidepsin, Apicidin, Trichostatin A, ACY-775, Tubastatin A, Tubacin, SKLB-23bb, HPOB, Curcumin, UF010, tc-H 106, Splitomicin, Raddeanin A, Depudecin, Tacedinaline, Isoguanosine, Parthenolide, Tinostamustine, Sodium Phenylbutyrate, Valproic Acid, Butyric acid, Phenylbutyric acid, 4-Phenylbutyric Acid, Divalproex Sodium, Sinapinic Acid, Suberohydroxamic Acid, Biphenyl-4-sulfonyl chloride, Thiophene Benzamide, Nicotinamide, Dihydrocoumarin, Naphthopyranone, and 2-Hydroxynaphthaldehyde;
(c) the RA agonist is selected from the group consisting of TTNPB, ATRA, 9-cis-Retinoic Acid, Adapalene, Tretinoin, WYC-209, DC271, Acitretin, Arotinoid, AGN190168, AGN205327, LGD1550, Ch55, AM580 (CD336), CD2081, BMS 753, Tamibarotene, AGN194078, AGN195183, AGN193836, CD2314, CD2019, CD666, C286, BMS 641, AC-55649, AC261066, KCL-286, CD1530, CD437, CD2325, BMS 189961, BMS 270394, BMS 961, Trifarotene, and Palovarotene;
(d) the FGFR (e.g., FGFR1) agonist is selected from the group consisting of FGF2, SUN11602, FGF1, FGF3, FGF4, FGF5, FGF6, FGF8, FGF10, FGF17, FGF19, FGF20, FGF21, FGF22, and FGF23;
(e) the glucocorticoid in steps (d) and (e) is independently selected from the group consisting of Hydrocortisone 21-hemisuccinate, Dexamethasone, Dexamethasone Acetate, Hydrocortisone, Cortisone, Prednisone, Prednisone Acetate, Meprednisone, Prednisolone, Methylprednisolone, Methylprednisolone Acetate, Fluprednisolone, Betamethasone, Paramethasone, Triamcinolone, Deflazacort, Fludrocortisone, Fludrocortisone Acetate, Deoxycorticosterone Acetate, Aldosterone, Beclometasone, Budesonide, Mometasone Furoate, Fluocinolone, Flunisolide, Fluorometholone, Fluticasone, Dagrocorat, Dagrocorat Hydrochloride, Mapracorat, Fosdagrocorat, GSK9027, GSK866, AZD2906, GW-870086, BAY 1003803, ZK 216348, LEO 134310, and RU28362;
(f) the CREB agonist is selected from the group consisting of Forskolin, cAMP, Dibutyryl cAMP, 8-Br-CAMP, cAMPS-Sp, and CW 008;
(g) the antioxidant in steps (d) and (e) is independently selected from the group consisting of N-Acetyl-L-Cysteine, Ascorbic Acid, Sodium Ascorbate, Glutathione, Ebselen, a-tocopherol, β-tocopherol, o-tocopherol, y-tocopherol, Lipoic Acid, Uric Acid, and Ubiquinol;
(h) the Wnt antagonist in each of steps (d) and (e) is independently selected from the group consisting of IWR-1-endo, C59, XAV939, WIKI4, JW55, JW74, NVP-TNKS656, LZZ-02, TC-E 5001, IWP2, IWP4, WNT974, CGX1321, ETC-159, RXC004, GNF-6231, WIF-1, Ipafricept, DKK1, BMD4503-2, Salinomycin, NSC 668036, FJ9, 3289-8625, LF3, CCT036477, CCT251545, MSAB, KY1220, KY02111, FH535, Triptonide, KYA1797K, iCRT3, iCRT5, iCRT14, PNU-74654, PKF118-310, Cardionogen 1, ICG-001, JW67, NLS-StAx-h, PRI-724, GNE-781, Capmatinib, NCB-0846, TAK715, Nitazoxanide, Vantictumab, OTSA-101, and Fz7-21;
(i) the TGF-β antagonist in each of steps (d) and (e) is independently selected from the group consisting of SB 431542, A 83-01, GW788388, SB505124, SB525334, TP0427736, RepSox, SD-208, Galunisertib, IN-1130, Ki 26894, LY2109761, LY2157299, LY550410, PF-03446962, TEW-7197, AP12009, AP11014, AP15012, ISTH0036, Fresolimumab, Lerdelimumab, GC1008, 2G7, 1D11, CAT-192, LY2382770, and LY3022859;
(j) the HGF agonist in each of steps (d) and (e) is independently selected from the group consisting of HGF, Dihexa, NK1, NK2, Fosgonimeton, and Terevalefim;
(k) the Notch antagonist is selected from the group consisting of DAPT, GSI-XX, BMS 299897, BMS 433796, BMS 906024, BMS 986115, Compound E, Compound W, Compound 18, DBZ, DFK-167, L-685458 LY 3039478, LY 411575, LY 450139, LY 900009, MK-0752, MRK 003, MRK 560, PF 3084014, PF 3084014 Hydrobromide, Z-IL-CHO, Avagacestat, Begacestat, JLK6, AL101, RO 4929097, FLI-06, Thapsigargin, CAD204520, Tangeretin, Bruceine D, 15D11, Enoticumab, Demcizumab, ABT-165, Navicixizumab, Marimastat, ZLDI-8, IMR-1, IMR-1A, CB-103, RIN1, Brontictuzumab, Tarextumab, and PF 06650808;
(l) the BMP antagonist is selected from the group consisting of LDN193189, Dorsomorphin, DMH-1, DMH-2, ML 347, LDN212854, LDN214117, K02288, Follistatin, Follistatin-like 1, Noggin, Chordin, Ventroptin, Twisted Gastrulation, Dan, Cerberus, PRDC, Dante, Caronte, Gremlin, and Sclerostin;
(m) the hepatocyte function enhancer is FH1 or FPH1.

3. The method of claim 2, wherein:
(a) the GSK3β inhibitor is CHIR99021;
(b) the HDAC inhibitor in step (c) is Vorinostat and/or the HDAC inhibitor in step (e) is Sodium Butyrate;
(c) the RA agonist is TTNPB;
(d) the FGFR (e.g., FGFR1) agonist is FGF2;
(e) the glucocorticoid in step (d) and/or step (e) comprises Hydrocortisone 21-hemisuccinate and Dexamethasone;
(f) the CREB agonist is Forskolin;
(g) the antioxidant in step (d) comprises N-Acetyl-L-Cysteine, Ascorbic Acid, and Sodium Ascorbate and/or the antioxidant in step (e) is Sodium Ascorbate;
(h) the Wnt antagonist in step (d) is IWR-1-endo and/or the Wnt antagonist in step (e) comprises IWR-1-endo and C59;
(i) the TGF-β antagonist in step (d) and/or step (e) is SB431542;
(j) the HGF agonist in step (d) comprises HGF and Dihexa and/or the HGF agonist in step (e) is Dihexa;
(k) the Notch antagonist is DAPT;
(l) the BMP antagonist is LDN193189;
(m) the hepatocyte function enhancer is FH1.

4. The method of claim 3, wherein:
(a) CHIR 99021 is present in the culture medium at a concentration of 100 nM-10 μM:
(b) Vorinostat is present in the culture medium at a concentration of 10 nM-5 μM and/or Sodium Butyrate is present in the culture medium at a concentration of 10 nM-5 μM;
(c) TTNPB is present in the culture medium at a concentration of 5 nM-2 μM;
(d) FGF 2 is present in the culture medium at a concentration of 500 pg/ml-500 ng/ml;
(e) Hydrocortisone 21-hemisuccinate is present in the culture medium at a concentration of 10 nM-100 μM and/or Dexamethasone is present in the culture medium at a concentration of 500 pM-5 μM;
(f) Forskolin is present in the culture medium at a concentration of 20 nM-2 mM;
(g) (i) N-Acetyl-L-Cysteine is present in the culture medium at a concentration of 500 nM-2 M, and/or
(ii) Ascorbic Acid is present in the culture medium at a concentration of 250 pg/ml-25 mg/ml, and/or
(iii) Sodium Ascorbate is present in the culture medium at a concentration of 200 pg/ml-20 mg/ml, and/or (iv) Sodium Ascorbate is present in the culture medium at a concentration of 200 pg/ml-20 mg/ml;
(h) IWR-1-endo is present in the culture medium at a concentration of 500 pM-10 μM and/or C59 is present in the culture medium at a concentration of 5 nM-100 μM;
(i) SB 431542 is present in the culture medium at a concentration of 10 nM-200 μM;
(j) HGF is present in the culture medium at a concentration of 100 pg/ml-10 μg/ml and/or Dihexa is present in the culture medium at a concentration of 500 pg/ml-1 μg/ml;
(k) DAPT is present in the culture medium at a concentration of 10 nM-100 μM;
(l) LDN 193189 is present in the culture medium at a concentration of 500 pM-2.5 μM;
(m) FH 1 is present in the culture medium at a concentration of 10 nM-2.5 mM.

5.-53. (canceled)

54. A culture medium for obtaining:
(a) endodermal cells, wherein the culture medium comprises a GSK3β inhibitor;
(b) midgut cells, wherein the culture medium comprises an HDAC inhibitor, an RA agonist, and an FGFR (e.g., FGFR1) agonist;
(c) hepatoblasts, wherein the culture medium comprises a glucocorticoid, a CREB agonist, an antioxidant, a Wnt antagonist, a TGF-β antagonist, and an HGF agonist;
(d) hepatocytes, wherein the culture medium comprises an HDAC inhibitor, a glucocorticoid, a CREB agonist, an antioxidant, a Wnt antagonist, a TGF-β antagonist, an HGF agonist, a Notch antagonist, a BMP antagonist, and a hepatocyte function enhancer.

55.-57. (canceled)

58. An isolated cell culture comprising:
(a) endodermal cells in a culture medium comprising a GSK3β inhibitor;
(b) midgut cells in a culture medium comprising an HDAC inhibitor, an RA agonist, and an FGFR (e.g., FGFR1) agonist;
(c) hepatoblasts in a culture medium comprising a glucocorticoid, a CREB agonist, an antioxidant, a Wnt antagonist, a TGF-β antagonist, and an HGF agonist; or
(d) 50 million to 50 billion hepatocytes in a culture medium comprising an HDAC inhibitor, a glucocorticoid, a CREB agonist, an antioxidant, a Wnt antagonist, a TGF-β antagonist, an HGF agonist, a Notch antagonist, a BMP antagonist, and a hepatocyte function enhancer.

59.-61. (canceled)

62. Endodermal cells generated by steps (a) and (b) of the method of claim 1.

63. Midgut cells generated by steps (a)-(c) of the method of claim 1.

64. Hepatoblasts generated by steps (a)-(d) the method of claim 1.

65. Hepatocytes generated by the method of claim 1.

66. An isolated cell culture comprising 50 million to 50 billion engineered hepatocytes generated by the method of claim 1.
